SEQUENCE LISTING (73 sequences)

SEQ ID NO: 1 - protein, 308 aa, Photinus pyralis
MGPVVEKIAELGKYTVGEGPHWDHETQTLYFVDTVEKTFHKYVPSQKKYTFCKVDKLVSFIIPLAGSPGRFVVSLEREIAILTWDGVSAAPTSIEAIVNVEPHIKNNRLNDGKADPLGNLWTGTMAIDAGLPVGPVTGSLYHLGADKKVKMHESNIAIANGLAWSNDLKKMYYIDSGKRRVDEYDYDASTLSISNQRPLFTFEKHEVPGYPDGQTIDEEGNLWVAVFQGQRIIKISTQQPEVLLDTVKIPDPQVTSVAFGGPNLDELHVTSAGLQLDDSSLDKSLVNGHVYRVTGLGVKGFAGVKVKL

SEQ ID NO: 2 - DNA, 927 bp, Photinus pyralis
atggggccagttgttgaaaaaattgcagaacttggcaagtatacggttggagaaggtcctcactgggatcatgaaactcagaccttatatttcgtcgacaccgtagagaaaacttttcataaatatgtaccttctcagaaaaaatacacgttttgtaaagtagataaactggtttctttcattattccccttgctggatcccctggccgttttgtagtcagtttggaacgtgaaatagccattcttacatgggatggcgttagtgctgcacctacaagcatagaagctattgttaatgtcgaaccacacattaaaaataacagactcaatgatggcaaagcagatccccttggcaatctatggacaggtacaatggctattgacgctggtctccccgtaggaccggtcactggcagtttatatcatttaggggctgataaaaaggtaaaaatgcacgagagcaacatagctatagcaaatgggctcgcgtggagtaatgatttgaagaaaatgtattatattgattcggggaaaagaagagtagacgagtacgattatgatgcttctacattatccatcagcaatcaacggccattatttacttttgaaaagcatgaagtgcctggatatccagatggtcaaacaattgatgaggagggtaatttatgggttgccgttttccaaggacagcgaattattaaaatcagtacccaacaaccggaagtgttactggataccgtaaaaataccagatcctcaggtcacctctgtagcatttggcggtccgaatttggatgaactgcatgtaacatctgctggtcttcagcttgacgacagttctttggacaaaagtttagttaatgggcacgtctacagagtaacaggtttaggcgtcaaaggtttcgcgggagttaaagtgaagctatga

SEQ ID NO: 3 - protein, 237 aa, Lampyris noctiluca
DKIPSLIIPISGCSNRFLITLDREIAILTWDGISSTPTSIKTIAVVDNEPGLESNRINDGKADHLGNLWAGTMDVNAFPTGPITGRLFSMNSTKDVRYYRTNIAVSNGIAWSKDLKKMYYIDSKTRVIDQYDFDASNRLISNRQTLFSLEKHQIPGFPDGQTIDTDDNLWVGVFEGNRVIKISTHIPETLLYTVNLPDSLITSVAFGGPNLDELYVTGGGNFLNGKVYKVSGLGVQG

SEQ ID NO: 4 - DNA, 863 bp, Lampyris noctiluca
gataaaataccgtctttgattattcccataagcggttgttcaaatcgttttcttattactttagaccgagaaatcgccatccttacttgggatggtataagttctacacccacaagtataaaaacaatcgctgtagttgacaatgagccaggtctggaaagtaatagaattaatgatggtaaagcagatcatcttggtaatctgtgggcaggtgaatgaagaaatctatagaactataacatgaaatccaacgcacctttttcaggcaccatggacgtaaatgcttttccaacgggaccaataactggtagattgtttagtatgaattctactaaagacgttagatactacagaacaaacatagcggtgtcaaacggaatcgcttggagtaaagatttgaagaaaatgtattatattgattccaagactagagtaatagatcaatacgattttgatgcttccaacagattgatcagtatacagtaaatgatttacattcgttgcatatttatgttgtttattttcaggtaatcgtcaaacattgtttagtctggaaaagcatcaaatcccgggatttcccgatggtcaaacaattgatactgatgataatttatgggttggtgtgttcgaaggaaacagagtgataaaaattagtactcatatacctgaaacattactgtatactgtaaaccttccagattcgctggtgagtaaaagtattgacattttcaaataatgtagatgtttattagataacctcggttgcttttggtggaccaaacttggatgaattgtacgtaacaggtggtggtaactttctgaatgggaaagtttataaagtatctggtctaggtgttcaaggt

SEQ ID NO: 5 - DNA, 25 bp, Artificial sequence
  Primer
atggggccagttgttgaaaaaattg

SEQ ID NO: 6 - DNA, 24 bp, Artificial sequence
  Primer
gcaatctatggacaggtacaatgg

SEQ ID NO: 7 - DNA, 25 bp, Artificial sequence
  Primer
gggctgataaaaaggtaaaaatgca

SEQ ID NO: 8 - DNA, 25 bp, Artificial sequence
  Primer
tcatagcttcactttaactcccgcg

SEQ ID NO: 9 - DNA, 17 bp, Artificial sequence
  Primer
gcactaacgccatccca

SEQ ID NO: 10 - DNA, 971 bp, Lampyris noctiluca
  misc_feature (102)..(102): n is uncertain
gtacgtctccaaatgacttcgtcatgagatgtgagcacttctggtatgtgttacgggtagttagatatcgcatttaacgacgttacctaacattttctactnatggggagtgttatatagtaaagttttgaataattttcgtaagctagtcacaggtctcgatagaagtgtaagctttaaccataaatgttaatgatattattccattcaattgagcttctataaaatcttcattttttggtttataatactgatgtgatcactgctagattacgttnttcgtagaattggcaatataattatttaagccgtaaatatttcatcagtgtaattgtcactacgcatcacttttttttgtctgtctagtacagcttacttccctcgttatttgtgtaatttcgcatagttatggataatatctagtattatctaaatttacctatngtgtattacgccatgtagttaaaggttgctgtattatcttatcatacattgaagttaatacacaagtaaaaaccttgatttttatagaaacctacgacagcattgcaacggcactagttaatagaaacagtggaagaagtggccaatatgcagtttggagaaggacctcattggaatgataagacacaaagtttatatttggtggaccttgtagataaatctattcacaaatatgtaccatctcttaaaagacaaacccatataaaagtaggcaagtattattacaacttgttatatccgagtcaaccaaataatgtaaaaaatttacgtcagtcccactataagatttaagaaattcataaatataaatttctatgctcattttcagataaaataccgtctttgattattgccataagcggttgttcaaatcgttttcttattactttagaccgagaaatcgccatccttacttgggatggcattagttctacacccacaagtatagaaacaatcgctgtagttga

SEQ ID NO: 11 - DNA, 1986 bp, Photinus pyralis
atggggccagttgttgaaaaaattgcagaacttggcaagtatacgagttggagaaggtcctcactgggatcatgaaactcagaccttatatttcgtcgacaccgtagagaaaacttttcataaatatgtaccttctcagaaaaaatacacgttttgtaaagtaggtaagaatcattgttcaaactatcacgcgtaaccgtttttacataagtacaatgtaaagtagctacgtacaccgtggatagtagcattgtatttataaacgaaatacaatggtagcattgtatttatatagtaggcactttatagttagaaataggggaagtattagccaagagtatacagggtggtgagttgcgtaacacattatagggatttcaatgtaaaattcataacatgcgattattggctccccaaattattttagagcaaaaatgcattaaggttttttgtagaaaaacattttttacatcaatcaaccgtattagttttaaaattttaagcaagtcaccaccctgtatatactctttggtattagtacaagatcccacataaggccaacctcgctcatctgtttactgagtgaaatacatctttgttagtaatcttttatgtgtaaacaaacacactaaaaaacttaagtcaagactatccttaaaatttctgtctgcttctattagatagagaggagacacacggaacaacacacgtttctctcccgcgcgtttcttaaaattacgacaaccgtgtgaaacaggacgttactcgtgacggcggtgtacgcattaatgttaagagagtgcggcgcactgaattttaattgagggtagtcttgacttaatttttttaggcaaccatttgcaatgtgtttgattacatattaaaattgctaaaacatgtaattcacttggtaaacagtagtcggtagggcgaataatgctgctatctattttctcttctatagcttattgagctgagtatgaagggtagaatagcatttagattagctaaagaatggcgtgagaatttaacccatctgtacataaactgaagttgatgccatttcagataaactggtttcattcattattccccttgctggatcccctggccgttttgtagtcagtttggaacgtgaaatagccattctcacgtgggatggcgttagtgctgcacctacgagcatagaagctattgttaatgtcgaaccccacattaaaaataacagactcaatgatggcaaagcagatcctcttggcaatctatggacaggtataactgaaatcgactaaccaaattataataattctagctcgaattctttaggtacaatggctattgacgctggtctccccgtaggaccggtcactggcagtttatatcatttaggggctgataaaaaggtaaaaatgcacgagagcaacatagctatagcaaatgggctcgcgtggagtaatgatttgaagaaaatgtattatattgattcgggaaaaagaagagtagacgagtacgattatgatgcttctacattatccatcagtaaacactatttaaatatattgcactattttttgttagatgaatattttcaggcaatcaacggccattatttacttttgaaaagcatgaagtgcctggatatccagatggtcaaacaattgatgcggagggtaatttatgggttgccgttttccaaggacagcgaattattaaaatcagtaccgaacaaccggaagtgttactggataccgtaaaaataccagatcctcaggtaaattaagtaactttgtgtattacattaattacctttggagtcaggtcacctctgttgcatttggcggtccgaatttggatgaactgtatgtaacatctgctggtcttcagcttgacgacagttcttttgacaaaagtttagttaatgggcacgtctacagagtaacaggtttaggcgtcaaaggtttcgcgggagttaaagtgaagctatga

SEQ ID NO: 12 - DNA, 20 bp, Artificial sequence
  Primer
aaraargtnaaratgcayga

SEQ ID NO: 13 - DNA, 20 bp, Artificial sequence
  Primer
aaraargtnaaaatgcacga

SEQ ID NO: 14 - DNA, 20 bp, Artificial sequence
  Primer
aaraargtnaagatgcacga

SEQ ID NO: 15 - DNA, 20 bp, Artificial sequence
  Primer
aaraargtnaaaatgcatga

SEQ ID NO: 16 - DNA, 20 bp, Artificial sequence
  Primer
aaraargtnaagatgcatga

SEQ ID NO: 17 - DNA, 17 bp, Artificial sequence
  Primer
athctnacntgggatgg

SEQ ID NO: 18 - DNA, 17 bp, Artificial sequence
  Primer
athctnacntgggacgg

SEQ ID NO: 19 - DNA, 20 bp, Artificial sequence
  Primer
ccrgartcdatrtartacat

SEQ ID NO: 20 - DNA, 20 bp, Artificial sequence
  Primer
ccrgartcdatatagtacat

SEQ ID NO: 21 - DNA, 20 bp, Artificial sequence
  Primer
ccrgartcdatgtagtacat

SEQ ID NO: 22 - DNA, 20 bp, Artificial sequence
  Primer
ccrgartcdatataatacat

SEQ ID NO: 23 - DNA, 20 bp, Artificial sequence
  Primer
ccrgartcdatgtaatacat

SEQ ID NO: 24 - DNA, 350 bp, Lampyris noctiluca
  misc_feature 182, 207, 208, 225, 282, 283, 329, 337, 342: n is uncertain
atcctgacgtgggatggcataagttctacacccacaagtatagaaacaatcgctgtagttgactatgagccaggtctggaaagtaatagaattaatgatggtaaagcagatcatcttggtaatctgtgggcaggtgaatgaagaaatctatagaactataacatgaaatccaacgcaccttnttcaggcaccatggacgtaaatgcnnatattccaacgggaccnataactggtagattgtttagtatgaattctactaaagacgttagatactacagaacnnacatagcggtgtcaaacggaatcgcttggagtaaagatctgaaganaatgtatnatatngactccgg

SEQ ID NO: 25 - DNA, 289 bp, Photinus pyralis
attcttacatgggatggcgttagtgctgcacctacaagcatagaagctattgttaatgtcgaaccacacattaaaaataacagactcaatgatggcaaagcagatccccttggcaatctatggacaggtacaatggctattgacgctggtctccccgtaggaccggtcactggcagtttatatcatttaggggctgataaaaaggtaaaaatgcacgagagcaacatagctatagcaaatgggctcgcgtggagtaatgatttgaagaaaatgtattatattgattggg

SEQ ID NO: 26 - DNA, 343 bp, Photinus pyralis
  misc_feature (241)..(241): n is uncertain
attctgacgtgggatggcgttagtgctgcacctacgagcatagaagctattgttaatgtcgaaccccacattaaaaataacagactcaatgatggcaaagcagatcctcttggcaatctatggacaggtataactgaaatcgactaaccaaattataataattctagctcgaattctttaggtacaatggctattgacgctggtctccccgtaggaccggtcactggcagtttatatcatntaggggctgataaaagggtagaaatgcacgagagcaacatagctatagcaaatgggctcgcgtggagtaatnatttgaagaaaatgtatnatattgattcgg

SEQ ID NO: 27 - DNA, 27 bp, Artificial sequence
  Primer
gatcatcttggtaatctgtgggcaggt

SEQ ID NO: 28 - DNA, 27 bp, Artificial sequence
  Primer
gcggtgtcaaacggaatcgcttggagt

SEQ ID NO: 29 - DNA, 27 bp, Artificial sequence
  Primer
acctgcccacagattaccaagatgatc

SEQ ID NO: 30 - DNA, 28 bp, Artificial sequence
  Primer
tctgctttaccatcattaattctattac

SEQ ID NO: 31 - DNA, 863 bp, Lampyris noctiluca
gataaaataccgtctttgattattcccataagcggttgttcaaatcgttttcttattactttagaccgagaaatcgccatccttacttgggatggtataagttctacacccacaagtataaaaacaatcgctgtagttgacaatgagccaggtctggaaagtaatagaattaatgatggtaaagcagatcatcttggtaacctgtgggcaggtgaatgaagaaatctatagaactataacatgaaatccaacgcacctttttcaggcaccatggacgtaaatgcttttccaacgggaccaataactggtagattgtttagtatgaattctactaaagacgttagatactacagaacaaacatagcggtgtcaaacggaatcgcttggagtaaagatttgaagaaaatgtattatattgattccaagactagagtaatagatcaatacgattttgatgcttccaacagattgatcagtatacagtaaatgatttacattcgttgcatatttatgttgtttattttcaggtaatcgtcaaacattgtttagtctggaaaagcatcaaatcccgggatttcccgatggtcaaacaattgatactgatgataatttatgggttggtgtgttcgaaggaaacagagtgataaaaattagtactcatatacctgaaacattactgtatactgtaaaccctccagattcgctggtgagtaaaagtattgacattttcaaataatgtagatgtttattagataacctcggttgcttttggtggaccaaacttggatgaattgtacgtaacaggtggtggtaactttctgaatgggaaagtttataaagtaactggtctaggtgttcaaggt

SEQ ID NO: 32 - DNA, 851 bp, Lampyris noctiluca
gataaaataccgtctttgattattcccataagcggttgttcaaatcgttttcttattactttagaccgagaaatcgccatccttacttgggatggcattagttctacacccacaagtatagaaacaatcgctgtagttgacaatgagccaggtcttgaaaataatagaattaatgatggtaaagcagatcatcttggtaatctgtgggcaggtgaatgaagaaatctataaaactataacatgaaatcaaacgcacctttttcaggcaccatggacgtaaatggatatccaacgggaccaataaccggtagattgtttagtataaattctactaaagacgttagatactacagaacaaacatagcggtgtcaaacggaatcgcctggagtaaagatttgaagaaaatgtattatattgattccatgactaaagtaatagatcaatacgattttgatgcttccaacagattgatcagtatacagtaaatgataaacattcgttacatatttatgttgtatattttcaggtaatcgtcaaaccttgtttagtcttgaaaataatcaaatcccgggatttccagatggtcaaacaattgatactgatgataatttatgggttgctgtgttcggaggaaacagagtgataaaaattagtactgatatacctgaaacattactgtatactgtaaacattccagattcgcaggtgagtaaatggattgacattttcaaataatgtagatgtttattagataacctcggttgcttttggtggaccaaacttggatgaattgtacgtaacaagtggtggtgggaaagtttataaggtaactggtctaggtgttcaaggt

SEQ ID NO: 33 - DNA, 863 bp, Lampyris noctiluca
gataaaataccgtctttgattgttcccataagcggttgttcaaatcgttttcttattactttagaccgagaaatcgccatccttacttgggatggtataagttctacacccacaagtataaaaacaatcgctgtagttgacaatgagccaggtctggaaagtaatagaattaatgatggtaaagcagatcatcttggtaatctgtgggcaggtgaatgaagaaatctatagaactataacatgaaatccaacgcacctttttcaggcaccatggacgtaaatgcttttccaacgggaccagtaactggtagattgtttagtatgaattctactaaagacgttagatactacagaacaaacatagcggtgtcaaacggaatcgcttggagtaaagatttgaagaaaatgtattatattgattccaagactagagtaatagatcaatacgattttgatgcttccaacagattgatcagtatacagtaaatgatttacattcgttgcatatttatgttgtttattttcaggtaatcgtcaaacattgtttagtctggaaaagcatcaaatcccgggatttcccgatggtcaaacaattgatactgatgataatttatgggttggtgtgttcgaaggaaacagagtgataaaaattagtactcatatacctgaaacattactgtatactgtaaaccttccagattcgctggtgagtaaaagtattgacattttcaaataatgtagatgtttattagataacctcggttgcttttggtggaccaaacttggatgaattgtacgtaacaggtggtggtaactttctgaatgggaaagtttataaagtatctggtctaggtgttcaaggt

SEQ ID NO: 34 - DNA, 851 bp, Lampyris noctiluca
gataaaataccgtctttgattattcccataagcggttgttcaaatcgttttcttattactttagaccgagaaatcgccatccttacttgggatggcattagttctacacccacaagtatagaaacaatcgctgtagttgacaatgagccaggtcttgaaaataatagaattaatgatggtaaagcagatcatcttggtaatctgtgggcaggtgaatgaagaaatctataaaactataacatgaaatcaaacgcacctttttcaggcaccatggacgtaaatggatatccaacgggaccaataaccggtagattgtttagtataaattctactaaagacgttagatactacagaacaaacatagcggtgtcaaacggaatcgcttggagcaaagatttgaagaaaatgtattatattgattccatgactaaagtaatagatcaatacgattttgatgcttccaacagattgatcagtatacagtaaatgataaacattcgttacatatttatgttgtatattttcaggtaatcgtcaaaccttgtttagtcttgaaaataatcaaatcccgggatttccagatggtcaaacaattgatactgatgataatttatgggttgctgtgttcggaggaaacagagtgataaaaattagtactgatatacctgaaacattactgtatactgtaaacattccagattcgcaggtgagtaaatggattgacattttcaaataatgtagatgtttattagataacctcggttgcttttggtggaccaaacttggatgaattgtacgtaacaagtggtggtgggaaagtttataaggtaactggtctaggtgttcaaggt

SEQ ID NO: 35 - DNA, 863 bp, Lampyris noctiluca
gataaaataccgtctttgattattcccataagcggttgttcaaatcgttttcttattactttagaccgagaaatcgccatccttacttgggatggcattagttctacacccacaagtatagaaacaatcgctgtagttgacaatgagccaggtcttgaaaataatagaattaatgatggtaaagcagatcatcttggtaatctgtgggcaggtgaatgaagaaatctataaaactataacatgaaatcaaacgcacctttttcaggcaccatggacgtaaatggatatccaacgggaccattaaccggtagattgtttagtataaattctactaaagacgttagatactacagaacaaacatagcggtgtcaaacggaatcgcttggagtaaagatttgaagaaaatgtattatattgattccatgactaaagtaatagatcaatacgattttgatgcttccaacagattgatcagtatacagtaaatgataaacattcgttacatatttatgttgtatattttcaggtaatcgtcaaaccttgtttagtcttgaaaataatcaaatcccgggatttccagatggtcaaacaattgatactgatgataatttatgggttggtgtgttcgaaggaaacagagtgataaaaattagtactcatatacctgaaacattactgtatactgtaaaccttccagattcgctggtgagtaaaagtattgacattttcaaataatgtagatgtttattagataacctcggttgcttttggtggaccaaacttggatgaattgtacgtaacaggtggtggtaactttctgaatgggaaagtttataaagtatctggtctaggtgttcaaggt

SEQ ID NO: 36 - DNA, 851 bp, Lampyris noctiluca
gataaaataccgtctttgattattcccataagcggttgttcaaatcgttttcttattactttagaccgagaaatcgccatccttacttgggatggcattagttctacacccacaagtatagaaacaatcgctgtagttgacaatgagccaggtcttgaaaataatagaattaatgatggtaaagcagatcatcttggtaatctgtgggcaggtgaatgaagaaatctataaaactataacatgaaatcaaacgcacctttttcaggcaccatggacgtaaatggatatccaacgggaccaataaccggtagattgtttagtataaattctactaaagacgttagatactacagaacaaacatagcggtgtcaaacggaatcgcttggagtaaagatttgaagaaaatgtattatattgattccatgactaaagtaataggtcaatacgattttgatgcttccaacagattgatcagtatacagtaaatgataaacattcgttacatatttatgttgtatattttcaggtaatcgtcaaaccttgtttagtcttgaaaagaatcgaatcccgggatttccagatggtcaamcaatagatactgatgataatttatgggttgctgtgttcggaggaaacagagtgataaaaattagtactgatatacctgaaacattactgtatactgtaaacattccagattcgcaggtgagtaaatggattgacattttcaaataatgtagatgtttattagataacctcggttgcttttggtggaccaaacttggatgaattgtacgtaacaagtggtggtgggaaagtttataaggtaactggtctaggtgttcaaggt

SEQ ID NO: 37 - DNA, 851 bp, Lampyris noctiluca
gataaaataccgtctttgattattcccataagcggttgttcaaatcgttttcttattactttagaccgagaaatcgccatccttacttgggatggcattagttctacacccacaagtatagaaacaatcgctgtagttgacaatgagccaggtcttgaaaataatagaattaatgatggtaaagcagatcatcttggtaatctgtgggcaggtgaatgaagaaatctataaaactataacatgaaatcaaacgcacctttttcaggcaccatggacgtaaatggatatccaacgggaccaataaccggtagattgtttagtataaattctactaaagacgttagatactacagaacaaacatagcggtgtcaaacggaatcgcttggagtaaagatttgaagaaaatgtattatattgattccatgactaaagtaatagatcaatacgattttgatgcttccaacagattgatcagtatacagtaaatgataaacattcgttacatatttatgttgtatattttcaggtaatcgtcaaaccttgtttagtcttgaaaagaatcaaatcccgggatttccagatggtcaaacaattgatactgatgataatttatgggttgctgtgttcggaggaaacagagtgataaaaattagtactgatatacctgaaacattactgtatactgtaaacattccagattcgcaggtgagtaaatggattgacattttcaaataatgtagatgtttattagataacctcggttgcttttggtggaccaaacttggatgaattgtacgtaacaagtggtggtgggaaagtttataaggtaactggtctaggtgttcaaggt

SEQ ID NO: 38 - DNA, 867 bp, Lampyris noctiluca
atgcagtttggagaaggacctcattgggatgataagacacaaagtttatatttggtggaccttgtagataaatctattcacaaatatgtaccatctcttaaaagacatacccatataaaagtagataaaataccgtctttgattattcccataagcggttgttcaaatcgttttcttattactttagaccgagaaatcgccatccttacttgggatggtataagttctacacccacaagtataaaaacaatcgctgtagttgacaatgagccaggtctggaaagtaatagaattaatgatggtaaagcagatcatcttggtaatctgtgggcaggcaccatggacgtaaatggatatccaacgggaccaataaccggtagattgtttagtataaattctactaaagacgttagatactacagaacaaacatagcggtgtcaaacggaatcgcttggagtaaagatttgaagaaaatgtattatattgattccatgactaaagtaatagatcaatacgattttgatgcttccaacagattgatcagtaatcgtcaaacattgtttagtcttgaaaataatcaaatcccgggatttccagatggtcaaacaattgatactgatgataatttatgggttgctgtgttcgaaggaaacagagtgataaaaattagtactcatatacctgaaacattactgtatactgtaaacattccagattcgctgataacctcggttgcttttggtggaccaaacttggatgaattgtacgtaacaggtggtggtaactttctgaatgggaaagtttataaagtatctggtctaggtgttcaaggtttacctgcaaataggattaaaatacataattaa

SEQ ID NO: 39 - protein, 288 aa, Lampyris noctiluca
MQFGEGPHWDDKTQSLYLVDLVDKSIHKYVPSLKRHTHIKVDKIPSLIIPISGCSNRFLITLDREIAILTWDGISSTPTSIKTIAVVDNEPGLESNRINDGKADHLGNLWAGTMDVNGYPTGPITGRLFSINSTKDVRYYRTNIAVSNGIAWSKDLKKMYYIDSMTKVIDQYDFDASNRLISNRQTLFSLENNQIPGFPDGQTIDTDDNLWVAVFEGNRVIKISTHIPETLLYTVNIPDSLITSVAFGGPNLDELYVTGGGNFLNGKVYKVSGLGVQGLPANRIKIHN

SEQ ID NO: 40 - DNA, 1125 bp, Lampyris noctiluca
  misc_feature (130)..(130): n is uncertain
atgcagtttggagaaggacctcattgggatgataagacacaaagtttatatttggtggaccttgtagataaatctattcacaaatatgtaccatctcttaaaagacatacccatataaaagtaggcaagnantatatacaacttgttatatccgagtcaaccaaataatgtaaaaaatttacgtcagtcccactataagatttaagaaattcataaatataaatttctatgctcattttcagataaaataccgtctttgattattcccataagcggttgttcaaatcgttttcttattactttagaccgagaaatcgccatccttacttgggatggcattagttctacacccacaagtatagaaacaatcgctgtagttgacaatgagccaggtcttgaaaataatagaattaatgatggtaaagcagatcatcttggtaatctgtgggcaggtgaatgaagaaatctataaaactataacatgaaatcaaacgcacctttttcaggcaccatggacgtaaatggatatccaacgggaccaataaccggtagattgtttagtataaattctactaaagacgttagatactacagaacaaacatagcggtgtcaaacggaatcgcttggagtaaagatttgaagaaaatgtattatattgattccatgactaaagtaataggtcaatacgattttgatgcttccaacagattgatcagtatacagtaaatgataaacattcgttacatatttatgttgtatattttcaggtaatcgtcaaaccttgtttagtcttgaaaagaatcgaatcccgggatttccagatggtcaaacaatagatactgatgataatttatgggttgctgtgttcggaggaaacagagtgataaaaattagtactgatatacctgaaacattactgtatactgtaaacattccagattcgcaggtgagtaaatggattgacattttcaaataatgtagatgtttattagataacctcggttgcttttggtggaccaaacttggatgaattgtacgtaacaagtggtggtgggaaagtttataaggtaactggtctaggtgttcaaggtttacctgcaaataggattaaaatacataattaa

SEQ ID NO: 41 - DNA, 867 bp, Lampyris noctiluca; identical to SEQ ID NO: 38

SEQ ID NO: 42 - DNA, 28 bp, Artificial sequence
  Primer
atgcagtttggagaaggacctcattggg

SEQ ID NO: 43 - DNA, 28 bp, Artificial sequence
  Primer
ctacttttatatgggtatgtcttttaag

SEQ ID NO: 44 - DNA, 23 bp, Artificial sequence
  Primer
ataaaataccgtctttgattatt

SEQ ID NO: 45 - DNA, 27 bp, Artificial sequence
  Primer
ctgcccacagattaccaagatgatctg

SEQ ID NO: 46 - DNA, 18 bp, Artificial sequence
  Primer
gcaccatggacgtaaatg

SEQ ID NO: 47 - DNA, 23 bp, Artificial sequence
  Primer
tgatcaatctgttggaagcatca

SEQ ID NO: 48 - DNA, 24 bp, Artificial sequence
  Primer
gtaatcgtcaaacattgtttagtc

SEQ ID NO: 49 - DNA, 23 bp, Artificial sequence
  Primer
cagcgaatctggaakgtttacag

SEQ ID NO: 50 - DNA, 25 bp, Artificial sequence
  Primer
ataacctcggttgcttttggtgacc

SEQ ID NO: 51 - DNA, 26 bp, Artificial sequence
  Primer
attatrtattttwatcctatttgcag

SEQ ID NO: 52 - DNA, 28 bp, Artificial sequence
  Primer
atggggccagttgttgaaaaaattgcag

SEQ ID NO: 53 - DNA, 24 bp, Artificial sequence
  Primer
tcatagcttcactttaactcccgc

SEQ ID NO: 54 - DNA, 39 bp, Artificial sequence
  Primer
gagctcccatgggccagtttggagaaggacctcattggg

SEQ ID NO: 55 - DNA, 37 bp, Artificial sequence
  Primer
gagctctcgagattatrtattttwatcctatttgcag

SEQ ID NO: 56 - DNA, 40 bp, Artificial sequence
  Primer
gagctctcgagttaattatrtattttwatcctatttgcag

SEQ ID NO: 57 - DNA, 34 bp, Artificial sequence
  Primer
gagctcccatggggccagttgttgaaaaaattgc

SEQ ID NO: 58 - DNA, 864 bp, Lampyris noctiluca
atgcagtttggagaaggacctcattgggatgataagacacaaagtttatatttggtggaccttgtagataaatctattcacaaatatgtaccatctcttaaaagacatacccatataaaagtagataaaataccgtctttgattattcccataagcggttgttcaaatcgttttcttattactttagaccgagaaatcgccatccttacttgggatggtataagttctacacccacaagtataaaaacaatcgctgtagttgacaatgagccaggtctggaaagtaatagaattaatgatggtaaagcagatcatcttggtaatctgtgggcaggcaccatggacgtaaatggatatccaacgggaccaataaccggtagattgtttagtataaattctactaaagacgttagatactacagaacaaacatagcggtgtcaaacggaatcgcttggagtaaagatttgaagaaaatgtattatattgattccatgactaaagtaatagatcaatacgattttgatgcttccaacagattgatcagtaatcgtcaaacattgtttagtcttgaaaataatcaaatcccgggatttccagatggtcaaacaattgatactgatgataatttatgggttgctgtgttcgaaggaaacagagtgataaaaattagtactcatatacctgaaacattactgtatactgtaaacattccagattcgctgataacctcggttgcttttggtggaccaaacttggatgaattgtacgtaacaggtggtggtaactttctgaatgggaaagtttataaagtatctggtctaggtgttcaaggtttacctgcaaataggattaaaatacataat

SEQ ID NO: 59 - protein, 288 aa, Lampyris noctiluca; identical to SEQ ID NO: 39

SEQ ID NO: 60 - DNA, 903 bp, Artificial sequence
  chimaeric LRE - ChimLREI
atggggccagttgttgaaaaaattgcagaacttggcaagtatacagttggagaaggtcctcactgggatcatgaaactcagaccttatatttcgtcgacaccgtagagaaaacttttcataaatatgtaccttctcagaaaaaatacacgttttgtaaagtagataaaataccgtctttgattattcccataagcggttgttcaaatcgttttcttattactttagaccgagaaatcgccatccttacttgggatggtataagttctacacccacaagtataaaaacaatcgctgtagttgacaatgagccaggtctggaaagtaatagaattaatgatggtaaagcagatcatcttggtaatctgtgggcaggcaccatggacgtaaatggatatccaacgggaccaataaccggtagattgtttagtataaattctactaaagacgttagatactacagaacaaacatagcggtgtcaaacggaatcgcttggagtaaagatttgaagaaaatgtattatattgattccatgactaaagtaatagatcaatacgattttgatgcttccaacagattgatcagtaatcgtcaaacattgtttagtcttgaaaataatcaaatcccgggatttccagatggtcaaacaattgatactgatgataatttatgggttgctgtgttcgaaggaaacagagtgataaaaattagtactcatatacctgaaacattactgtatactgtaaacattccagattcgctgataacctcggttgcttttggtggaccaaacttggatgaattgtacgtaacaggtggtggtaactttctgaatgggaaagtttataaagtatctggtctaggtgttcaaggtttacctgcaaataggattaaaatacataat

SEQ ID NO: 61 - protein, 301 aa, Artificial sequence
  chimaeric LRE - ChimLREI
MGPVVEKIAELGKYTVGEGPHWDHETQTLYFVDTVEKTFHKYVPSQKKYTFCKVDKIPSLIIPISGCSNRFLITLDREIAILTWDGISSTPTSIKTIAVVDNEPGLESNRINDGKADHLGNLWAGTMDVNGYPTGPITGRLFSINSTKDVRYYRTNIAVSNGIAWSKDLKKMYYIDSMTKVIDQYDFDASNRLISNRQTLFSLENNQIPGFPDGQTIDTDDNLWVAVFEGNRVIKISTHIPETLLYTVNIPDSLITSVAFGGPNLDELYVTGGGNFLNGKVYKVSGLGVQGLPANRIKIHN

SEQ ID NO: 62 - protein, 309 aa, Luciola cruciata
MAPTVEQIVELGTYLLAESPHWDDETQSLYFVDIVGRSVNKYVPTTKTHTQLKFDKNPSFIIPVKGCSDRFIVSLEREINLLTWDGASSAPSKIEKIAVFDNTPEKSENRLNDGKADPLGNLWAGTMNMGSDHTTGTPVRGTLSSLSNKQVKEHVSEVCISNGLAWSKDLKKFYYIDSAVRQVDQFDFDAKNLSLSNRQPLFTFDKHGIMGSPDGQTIDAEGNLWVATCQGDKVLKIDTSTPETLLGIVEIPEHQVTSVCIGGAELNVLYVTTASIKLPGADETKPMKGAIYKVTGLGVKGLPGDRVKL

SEQ ID NO: 63 - protein, 307 aa, Luciola lateralis
MSPVIEQITEVDNFQIGEGPHWDTETQSLYFVDILEKSIHKYVPSTKQHTKMILNKRPSFIIPIKETSDRFVISLERDICVLTWDGVSATPSHLETIVTVDTGIEGNTFNDGKADAFGNLWAGTLYSKFDIEKQGPNTGTLYSLSNKQLRKHISNIFLSNGLAWNKDSKKFYFIDSNKRTIDQFDYDSENLIISNCQPLFTLDKHGIQGLPDAQTIDENDNLWVAIVRGGKVINIGTKQPESLLGVINMPESLITSVCFGGSKLDELYVTTSGIKEYETDSTKLVKGGLYRVTGLGVKGLPAHRFSL

SEQ ID NO: 64 - DNA, 958 bp, Lampyris noctiluca
  misc_feature (389)..(389): n is uncertain
atcgcatttatcgacgttcctaacattttactacttttggggagtgttatattgtaaagttttgaataattttcgtaagctagtcacaggtctcgatagaagtgtaagctttaaccataaatgttaatgatattattccattcaattgagcttctataaaatcttcattttttggtttataatgctgacgtgatcactgctagattacgttattcgtagaatttgcaatataattatttaagccgaaatatttcatcaatgagtacctcagtgtaattgtcactacgcatcacttttttttgtctgtctagtacagcttacttccctcgttatttgtgtaatttcgtatagttatggataatatctagtattatctaaatttacctatngtgtattacgtcatgtagttaaaggttgctgtattatcatacatagaagttaatacacaagtaaaaaccttgatttttatagaaacctacgacagcattgcaacggcactagttatagaaacagttgaagaagtggccaatatgcagtttggagaaggacctcattgggatgataagacacaaagtttatatttggtggaccttgtagataaatctattcacaaatatgtaccatctcttaaaagacatacccatataaaagtaggcaagtattattacaacttgttatatccgagtcaaccaaataatgtaaaaaatttacgtcagtcccactataagatttaagaaattcataagtataaatttctatgctcattttcagataaaataccgtctttgattattgccataagcggttgttcaaatcgttttcttattactttagaccgagaaatcgccatccttacttgggatggcattagttctacacccacaagtatagaaacaatcgctgtagttgacaatgagccaggtcttgaaagtaatagaattaatgatggtaaagcaga

SEQ ID NO: 65 - DNA, 956 bp, Lampyris noctiluca
aaattacttagtcgtcttttgtaagtttatgaaaaagttgcatttttattaatttgaattttgaataattttcgtaagctagtcacaggtctcgatagaagtgtaaactttaaccataaatgttaataatattattctattcaactgagcttctataaaatcttcatttttggtttataatactgatgtaatctctgctagattacgctttggtagaatggcaatataattaataattatttaattctaaatacttgatcaatgagtacctccgtgtaattgtcactacgcatcactttttttgtctgtctagtacagcttacttccctcgttatttgtgtaatttcgtgtagttatggataatatctagtcttatctaaatttatgtactacgtcatgtagttaaagattgctgtatcatcatacattaaagttaaatgcaaaagtaaaaacattgatttttatagaaaactacggcagcattgcaacggcactagttatagaaacagttgaagaattgggcacttttaagactggagaaggacctcattgggatgataaaacacaaagtttatatttggtggaccttgtagataaatctattcacaaatatgtaccatctcttaaaagacatacccatataaaagtaggcaagtattattacaacttgttatatccaagtcaaccaaataatgtaaaaaatttacgtcagtcccacattcataaatataaatatatcataaatataaatttctatgctcattttcagataaaataccgtctttgattattcccataagcggttgttcaaatcgttttcttattactttagaccgagaaatcgccatccttacttgggatggtataagttctacacccacaagtataaaaacaatcgctgtagttgacaatgagccaggtcttgaaagtaatagaattaatgatggtaaagcaga

SEQ ID NO: 66 - protein, 300 aa, Photinus pyralis
MGPVVEKIAELGKYTVGEGPHWDHETQTLYFVDTVEKTFHKYVPSQKKYTFCKVDKLVSFIIPLAGSPGRFVVSLEREIAILTWDGVSAAPTSIEAIVNVEPHIKNNRLNDGKADPLGNLWTGTMAIDAGLPVGPVTGSLYHLGADKKVKMHESNIAIANGLAWSNDLKKMYYIDSGKRRVDEYDYDASTLSISNQRPLFTFEKHEVPGYPDGQTIDEEGNLWVAVFQGQRIIKISTQQPEVLLDTVKIPDPQVTSVAFGGPNLDELHVTSAGLQLDDSSLDKSLVNGHVYRVTGLGVKG

SEQ ID NO: 67 - DNA, 292 bp, Artificial sequence
  pET-28a-c(+) cloning/expression region
agatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccgaattcgagctccgtcgacaagcttgcggccgcactcgagcaccaccaccaccaccactgagatccggctgctaacaaagccc

SEQ ID NO: 68 - protein, 58 aa, Artificial sequence
  pET-28a-c(+) cloning/expression region
MGSSHHHHHHSSGLVPRGSHMASMTGGQQMGRGSEFELRRQACGRTRAPPPPPLRSGC

SEQ ID NO: 69 - DNA, 94 bp, Artificial sequence
  pET-28a-c(+) cloning/expression region
ggtcgggatccgaattcgagctccgtcgacaagcttgcggccgcactcgagcaccaccaccaccaccactgagatccggctgctaacaaagccc

SEQ ID NO: 70 - protein, 23 aa, Artificial sequence
  pET-28a-c(+) cloning/expression region
GRDPNSSSVDKLAAALEHHHHHH

SEQ ID NO: 71 - DNA, 93 bp, Artificial sequence
  pET-28a-c(+) cloning/expression region
ggtcggatccgaattcgagctccgtcgacaagcttgcggccgcactcgagcaccaccaccaccaccactgagatccggctgctaacaaagccc

SEQ ID NO: 72 - protein, 31 aa, Artificial sequence
  pET-28a-c(+) cloning/expression region
GRIRIRAPSTSLRPHSSTTTTTTEIRLLTKP

SEQ ID NO: 73 - DNA, 89 bp, Artificial sequence
  pET-28a-c(+) cloning/expression region
gaaaggaagctgagttggctgctgccaccgctgagcaataactagcataaccccttggggcctctaaacgggtcttgaggggttttttg

(19) United States
(12) Patent Application Publication
(10) Pub. No.: US 2005/0037355 A1
(43) Pub. Date: Feb. 17, 2005

(54) Signal system and elements used therein

(76) Inventors: John Cavendish Day, Oxford (GB); David James Squirrell, Wiltshire (GB); Mark John Bailey, Oxford (GB); Peter John White, Wiltshire (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/495,715

(22) PCT Filed: Nov. 13, 2002
(86) PCT No.: PCT/GB02/05120

(30) Foreign Application Priority Data
Nov. 14, 2001 (GB) 0127292.1
Mar. 6, 2002 (GB) 0205201.7

(51) Int. Cl. C12Q 1/68; C12Q 1/66

(52) U.S. Cl. 435/6; 435/8

(57) ABSTRACT

A method for measuring intracellular ATP and/or gene expression, which method comprises transforming a cell with a construct which encodes a luciferase and a luciferin recycling protein, introducing luciferin into said cell, and monitoring biolominescent signals from said cell. Novel luciferin recycling proteins are also described and these, together with their use in the production of optically active enzyme substrates are claimed.

The present invention relates to applications for a bioluminescent signalling system, in particular comprising luciferase and luciferin, as well as to certain novel genes and proteins used in the process, and to the production of elements used in the system.

Bioluminescent signalling systems are well known and have a wide range of applications in biotechnology, in particular in the fields of detection of micro-organisms, or as physiological reporters in investigations of cell activity.

One of the commonest bioluminescent signalling systems utilises a combination of the enzyme luciferase, found in nature in organisms such as fireflies and glow-worms, and the enzyme substrate, luciferin. In the presence of adenosine triphosphate (ATP) found in all cells, luciferase oxidises luciferin to produce oxyluciferin and cysteine as well as a bioluminescent signal, which may be monitored for example using a luminometer.

The signalling reaction can be represented as follows: 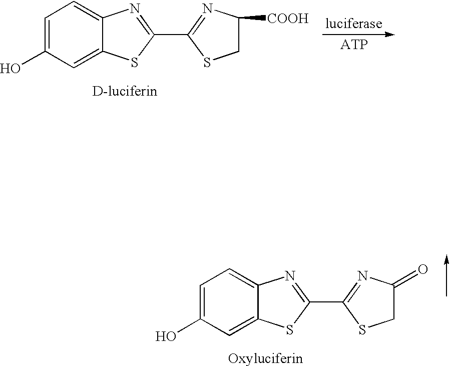
where the dotted arrow indicates a bioluminescent signal.

Oxyluciferin is however an inhibitor of the reaction, which means that any signal generated in such a system is very short lived. Furthermore, it is necessary to provide a considerable excess of D-luciferin to any reaction system to ensure that an adequate and measurable signal is generated.

Examples of assays for the detection of microorganisms and the like, which use such a system include those described for example in WO 94/17202 and WO 96/02665.

Intracellular ATP concentrations can vary 10-fold or more depending upon a cell's state of health or developmental stage. It is of great value to be able to measure fluctuations in intracellular ATP levels as a means of investigating e.g. the effects of drugs, toxins, hormones, environmental agents or disease on cells.

Various methods for analysing the concentration of ATP in vivo are suggested in the art. For instance, in Dementieva et al (1996) Biochemistry (Moscow) Vol 61, No. 7., the intracellular concentration of ATP was measured in E. coli by calculating the total amount of ATP present using a recombinant luciferase, and dividing by an estimated total cell volume.

Such an indirect approach can at best produce only an estimate of the actual ATP concentration.

Luciferase has sometimes been used as a marker for gene expression (in vivo) where its production in a cell is linked to a particular genetic control element. Luciferin is added exogenously and intracellular ATP concentrations, under almost all conditions, will be such that the enzyme is saturated. Thus the switching on of gene expression is signalled by light that is emitted in a quantitative manner according to the amount of active luciferase that is generated.

It is generally the concentration of luciferase which is measured; this concentration is then correlated with a different event e.g. the efficiency of a promoter. It is known that using luciferases with reduced Michaelis-Menten constant Km for ATP (see e.g. WO 96/22376) ensures that changes in the ambient [ATP] does not interfere with the assay.

A mutant luciferase, which may be particularly useful in cellular ATP assays conducted in vivo because of the higher than normal Michaelis-Menten constant Kₘ, is described in WO 98/46729. By using luciferases which have an increased Kₘ compared with those already known in the art, the possibility of using these enzymes to measure steady state ATP concentrations over a wide range. This is because, generally speaking, the relationship between enzyme velocity (V, as measured by light intensity) and substrate concentration (of ATP, where luciferin is in excess) is as follows:
V=Vₘ. [ATP]/Kₘ+[ATP]

It can therefore be seen that only when the Km is greater than (or of a similar order as) the ambient [ATP] will there be a degree of proportionality between changes in [ATP] and changes in light intensity. Where the Kmis much less than the ambient [ATP], any changes in [ATP] will not tangibly effect the measured light intensity. Clearly the more sensitive the light detection is, the smaller the measurable changes in ‘V’ can be, and the smaller the Km can be with respect to the [ATP] range being assessed.

For certain applications, e.g. in vivo measurements, it may be advantageous to have a luciferase wherein the Kₘ is of the order of between 400 μm to 1.4 mM e.g. 500 μm, 600 μm, 1 mM etc. However, as can be appreciated from the discussion above, the main criterion is that the Kₘ is not much less than the expected [ATP] range to be assessed.

A particular expected [ATP] range which is important for physiological assays of blood cells is between 300 pm and 1 mM, or more particularly 380 μm and 620 μm, (cf. Sigma Diagnostic Kit, Catalog No. 366 discussed above). For other mammalian cells such as hepatocytes, the [ATP] range is 2.5 mM-6 mM (see Dementieva et al (1996) discussed above). Use of the recombinant luciferases such as those described in WO 96/22376 are particularly suitable for continuous assays in these ranges.

In all such assays, however, as well as those used as physiological reporters of cell health, for example during screening for drugs etc., the cell is suitably transformed so that it expresses a luciferase enzyme. However, as mentioned above, luciferin must be applied exogenously. Unfortunately, luciferin is only cell-permeable at pH<5, which is not generally regarded as being a physiologically acceptable pH. Thus addition of this reagent to a conventional cellular assay is not easy. In order to monitor the ATP levels of the cell over a protracted period, it is necessary to maintain this low pH over that period, which could in itself have a detrimental effect on the cells. This limits application of assays of this type, in particular in high-throughput screening where robust methodology is required.

Furthermore, although luciferase enzyme may be produced using recombinant DNA technology, D-luciferin is generally produced synthetically, as a mixture of the desired D-isomer and the unwanted L-isomer, which must be separated prior to use. This is a wasteful procedure.

U.S. Pat. No. 5,814,504 describes a 40 kD protein, isolated from firefly species, which produces firefly D-luciferin when combined with oxyluciferin and D-cysteine. This protein is said to be useful in improving the durability of the luminescent signal from the luciferase/luciferin reaction system and in reducing the amount of luciferase and luciferin used in the reaction. Methods of producing firefly luciferin using this protein are also described. The amino acid sequence of this protein and the corresponding mRNA from Photinus pyralis are available on the NCBI database as Accession Number BAB60700 and Accession Number AB062786 respectively.

The sequence of other luciferase recycling enzymes, derived from Luciola cruciata and Luciola lateralis have also been published (see BAB85479 and BAB85478 respectively).

The applicants have developed further applications for proteins of this type, and nucleic acids encoding them, and have found further novel examples of proteins with related activity, obtainable from glow worms.

According to the present invention there is provided a method for measuring intracellular ATP and/or gene expression, which method comprises transforming a cell with a construct which encodes a luciferase and a luciferin recycling protein, introducing luciferin into said cell, and monitoring bioluminescent signals from said cell.

The expression “luciferin recycling protein” or LRE refers to proteins which convert oxyluciferin and cysteine to luciferin. An example of such a protein is that described in U.S. Pat. No. 5,814,504 but the applicants have cloned and sequenced further examples and these form a further aspect of the invention, as will be explained further below.

The method of the invention can be used to monitor intracellular ATP levels. Alternatively or additionally it may be used to monitor expression of either the luciferase gene or the luciferin recycling gene since the signal produced will be related to expression levels of either of these, although more directly related to the expression of luciferase.

The method of the invention has significant advantages in allowing in vivo gene expression to be reported with a stable light output. A single luciferin charge may be introduced into the cell at the beginning of the cell assay, during a brief exposure to pH 5. Thereafter the cell can be restored to physiological pH during the assay. Although the luciferin will be used rapidly in the system, the oxyluciferin produced will be converted back to D-luciferin by the action of the luciferin recycling protein expressed within the cell, and cysteine, in particular D-cysteine, present within the cell.

As a result, a relatively stable light output is achievable, which provides a signal that is easier to read. There may be particular advantages to a system of this type.

The luciferase enzyme and the luciferin recycling protein may be expressed within the cell as two separate proteins. In this case, the construct used to transform the cell may be a two part construct, one part containing the gene encoding the luciferase enzyme, and the second part containing the gene encoding the luciferin recycling protein. Preferably the luciferase enzyme and the luciferin recycling protein are expressed together as a fusion protein. By genetically linking the luciferase activity and the recycling activity, the kinetics of the reaction, (either in-vivo or in-vitro) become more favourable. Such fusion proteins form a further aspect of the invention.

If necessary, the cellular content of D-cysteine may be increased, for example by further transforming the cell so that it expresses an L-cysteine racemase.

In a preferred embodiment, the cell is transformed such that it expresses two luciferase enzymes, one with a relatively high Km value and one with a relatively low Kₘ value. Suitably these have outputs at different wavelengths, which may be distinguished. In this system, the useful range of the assay may be extended. Alternatively or additionally, it allows a ratiometric assay to be conducted, where the activity of the high Kₘ luciferase is compared with that of the low Kₘ luciferase. In this way, cellular physiology may be continuously monitored, for example so that cell poisons or other adverse effects of a sample being screened, could be quickly detected.

Examples of luciferase mutants with a relatively low Kₘ value are described in WO 96/22376, and these may be used in the context of the invention.

Novel constructs used in transforming the cells for use in the method of the invention form a further aspect of the invention. Thus, the invention further provides a DNA construct comprising (i) a nucleic acid sequence which encodes a luciferase enzyme, and (ii) a nucleic acid sequence which encodes a luciferin recycling protein. These elements are preferably under the control of a suitable promoter. Where the elements are expressed as two proteins, the nucleic acid sequence which encodes the luciferase enzyme will be under the control of a first promoter, and the nucleic acid sequence which encodes the luciferin recycling protein will be under the control of a second promoter.

Preferably, however, the nucleic acid sequences of these elements are linked so as to express a fusion protein of the luciferase and the luciferin recycling enzyme. In this case, the nucleic acid sequence is under the control of a single promoter.

As described above, the construct may suitably comprise one or more additional components selected from (iii) a nucleic acid sequence which encodes a further luciferase enzyme; and (iv) a nucleic acid sequence which encodes an L-cysteine racemase enzyme. Elements (iii) and (iv) are suitably under the control of third and fourth promoters respectively.

The construct is suitably in the form of one or more vectors that may be used in cell transformation. Where the elements of the construct are present on more than one vector, these may be used to co-transform the target cell.

Cells transformed in this way may be eukaryotic or prokaryotic. For cellular assays, it might be envisaged that eukaryotic cells, such as mammalian or plant cells would be required. Vectors and promoters are selected such that they are active in the target cell type, as is conventional in the art.

Nucleic acids used in the constructs of the invention encode proteins having the specified activity. It may be preferred that at least some of the codons used in these nucleic acids are “optimised” for the target cell species, as is conventional in the art. For example, particular cell types will express more effectively nucleic acids with a particular percentage GC content, and as a result of the degeneracy of the genetic code, codons may be selected so that the nucleic acids have a % GC content resembling this.

Examples of nucleic acid sequences of elements (i) and/or (iii) above are well known in the art. They suitably encode luciferases having desired properties such as thermostability, Km values and colorimetric properties. Examples of mutant luciferases which are suitably encoded by the nucleic acids used in the constructs of the invention are described in EP-A-0528448, WO95/25798, Wo 96/22376, WO 98/46729, WO 00/24878, WO 01/31028, WO 99/14336 and WO 01/20002.

Examples of suitable L-cysteine racemase enzymes (element (iv) above) are known in the art. For example, the enzyme amino acid racemase, with low substrate specificity, from Pseudomonas putida (designated EC 5.1.1.10 on the EcoCyc database) is known to catalyse the conversion of L-amino acids to D-amino acids.

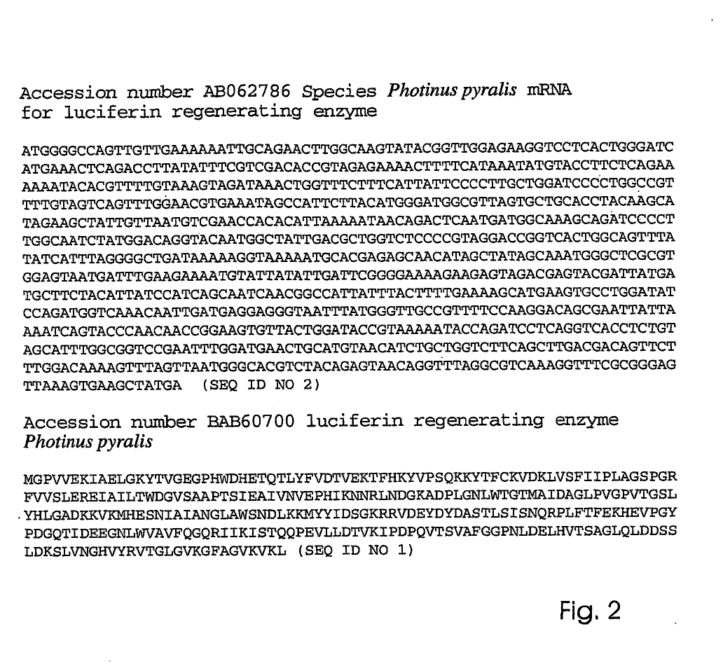

An example of a nucleic acid which may be used in element (ii) of the construct of the invention encodes a protein of SEQ ID NO 1 as illustrated hereinafter in FIG. 2, or a luciferin recycling fragment or variant thereof. This protein is obtainable from Photinus pyralis.

Other known examples of luciferin recycling enzymes are proteins obtainable from Luciola species such as Luciola cruciata and Luciola lateralis. Particular examples of such sequences are given hereinafter as SEQ ID NO 62 and 63. Thus the nucleic acids included in the constructs of the invention may encode proteins of SEQ ID NOS 62 or 63, or luciferin recycling fragments thereof, or variants of any of these.

As used herein, the term “fragment” refers to one or more portions of the basic sequence which has the required enzyme activity. These may be deletion mutants. Generally speaking the portions will comprise at least 12 and preferably at least 20 amino acids of the basic sequence.

The expression “variant” includes allelic variants and variants found at different loci as a result of the presence of multiple gene copies. In addition, the term “variant” includes sequences of amino acids or nucleic acids, which encode them, which differ from the base amino acid sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 60% homologous, preferably at least 75% homologous, and more preferably at least 90% homologous to the base sequence. Homology in this instance can be judged for example using the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

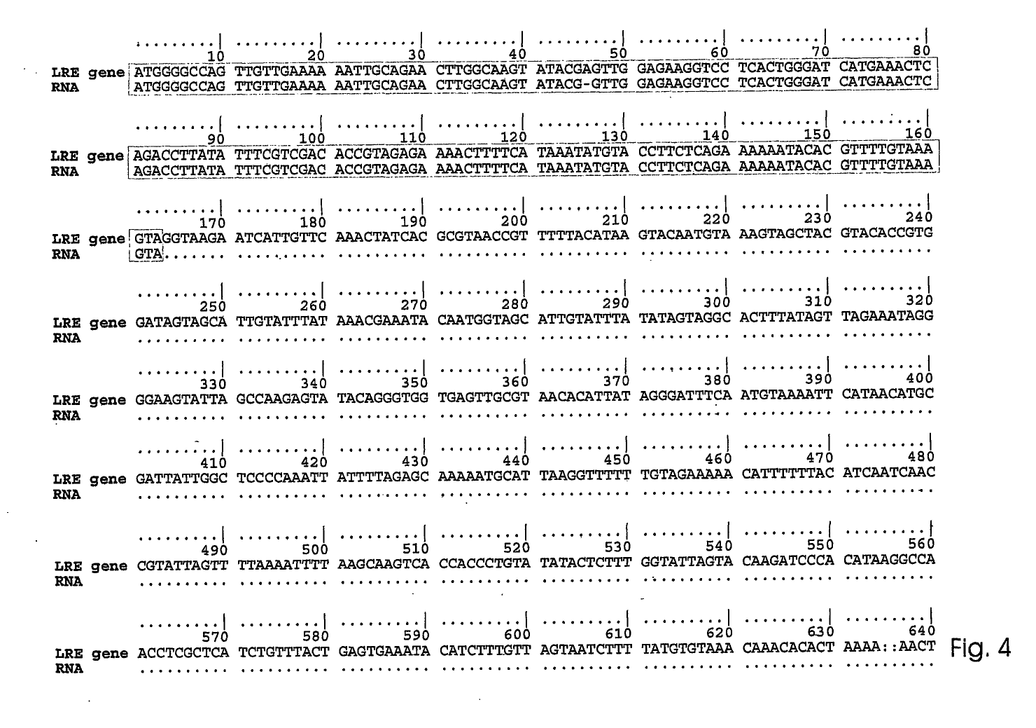

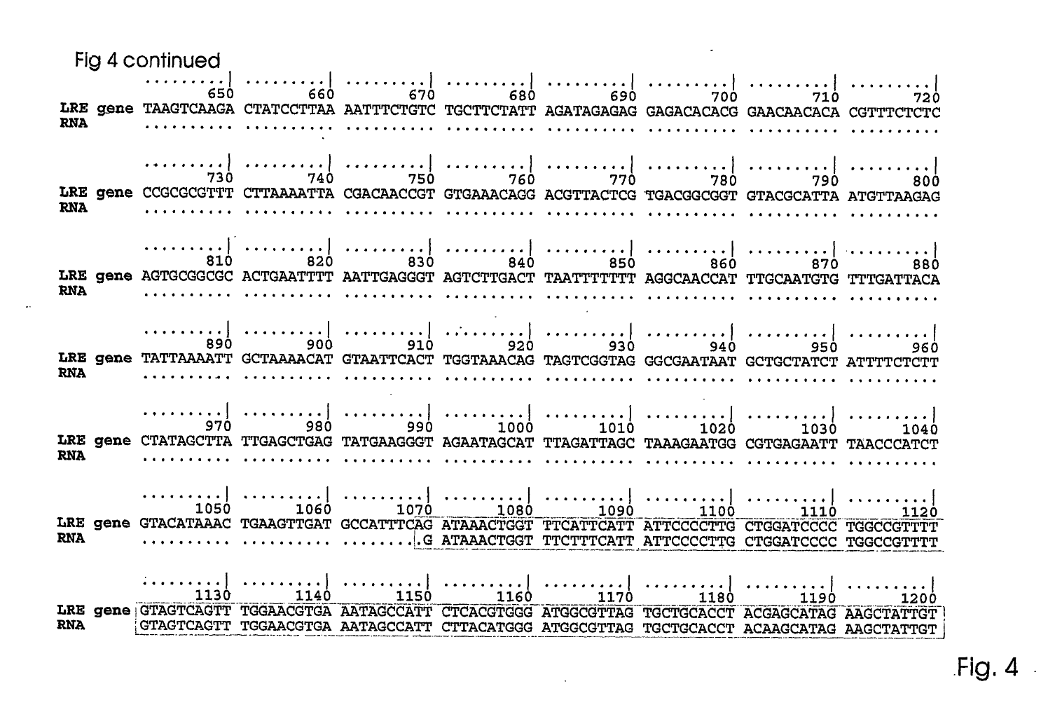

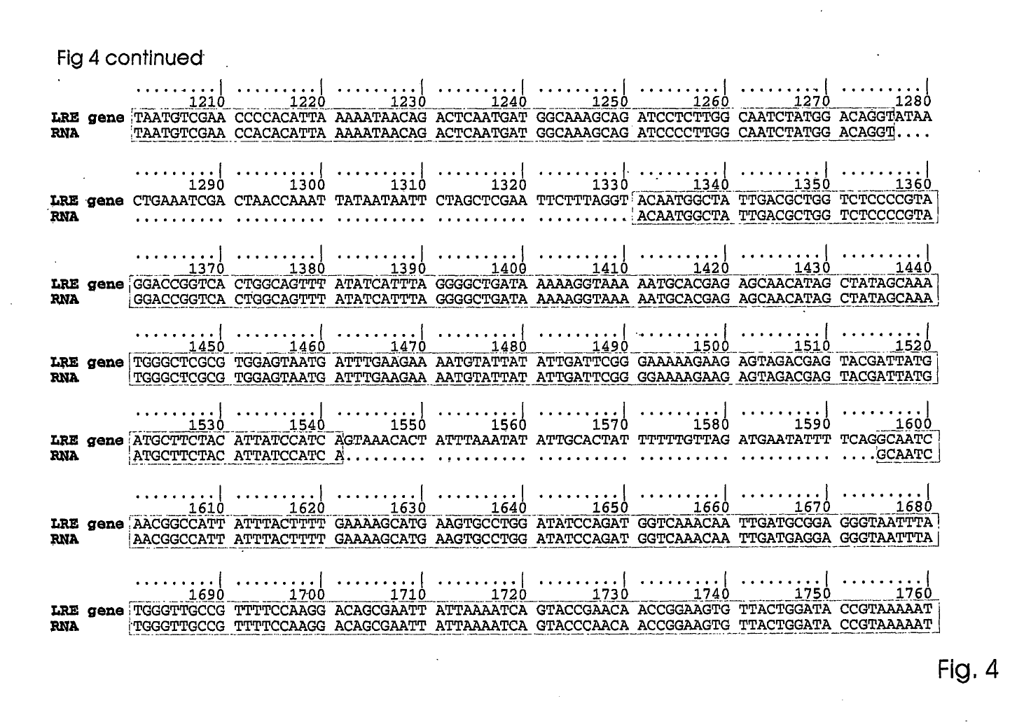

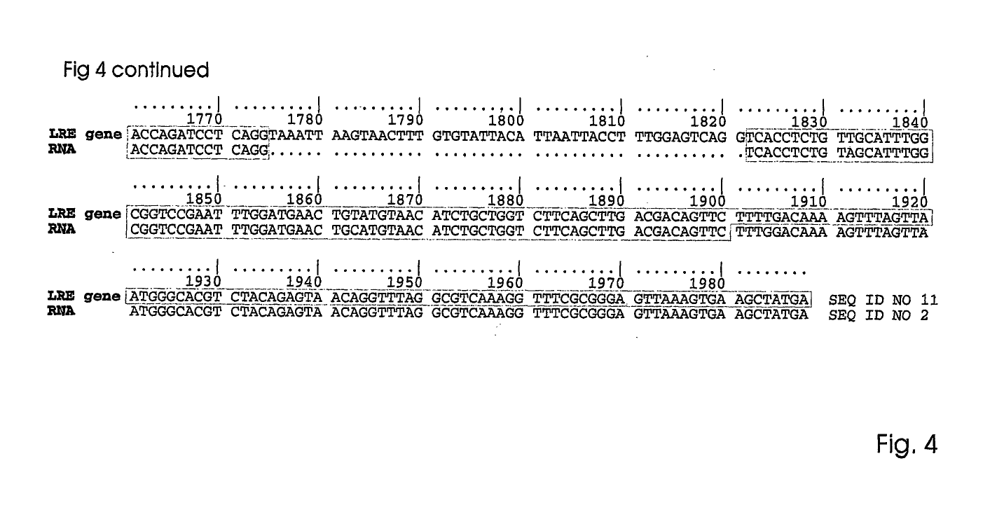

A particular example of a nucleic acid which encodes SEQ ID NO 1 is the mRNA sequence shown as SEQ ID NO 2 in FIG. 2 hereinafter. An example of a suitable genomic sequence, which encodes the protein of SEQ ID NO 1, is SEQ ID NO 11 as illustrated in FIG. 4 hereinafter.

In an alternative embodiment, the construct of the invention includes as element (ii) a nucleic acid which encodes a luciferin recycling protein obtained from a glow worm species such as Lampyris noctiluca, or luciferin recycling fragements thereof, or variants of any of these.

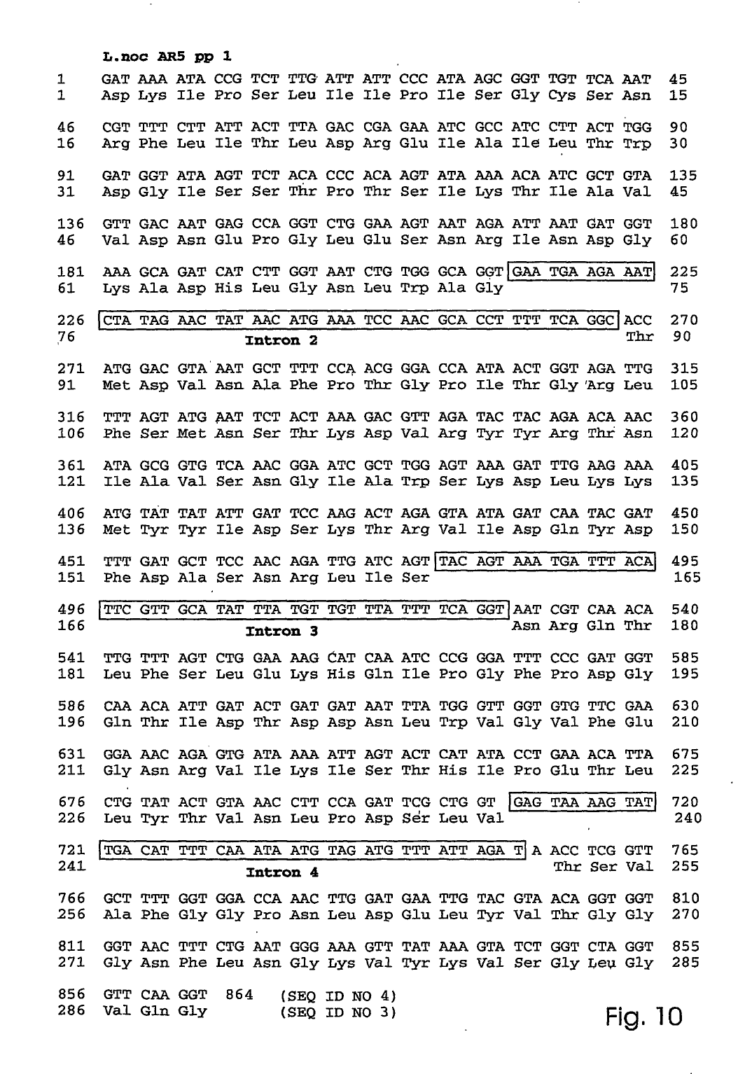

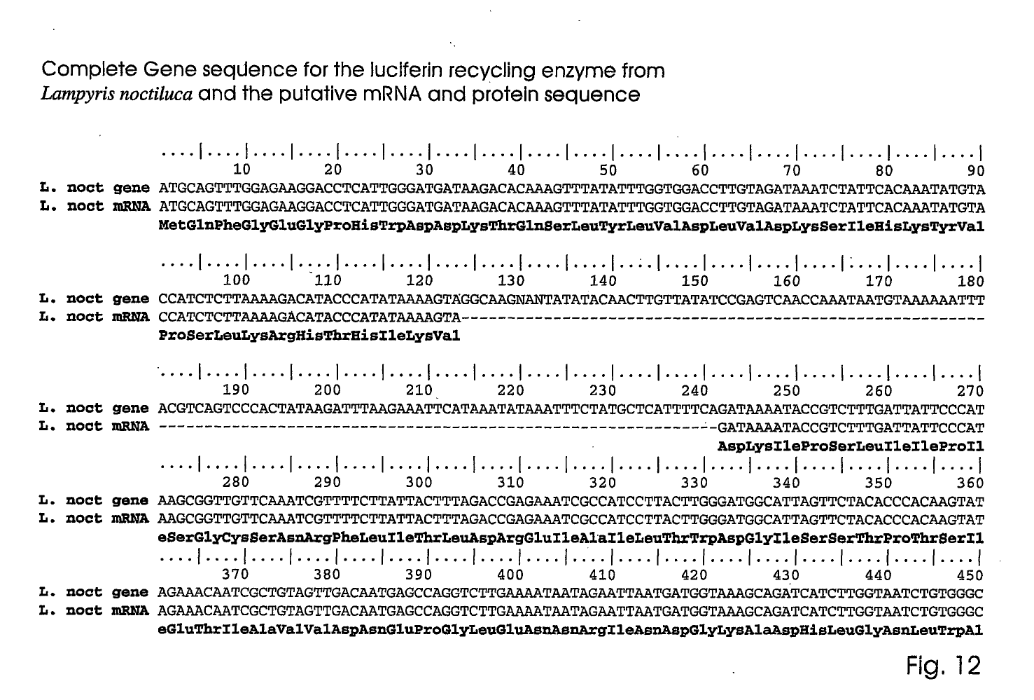

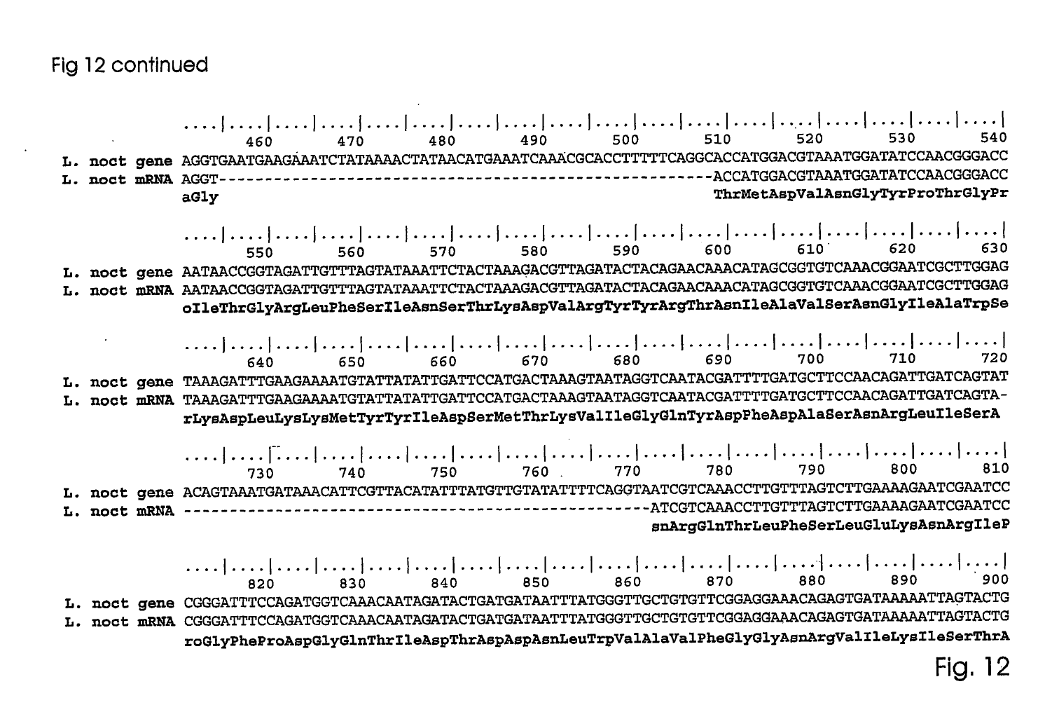

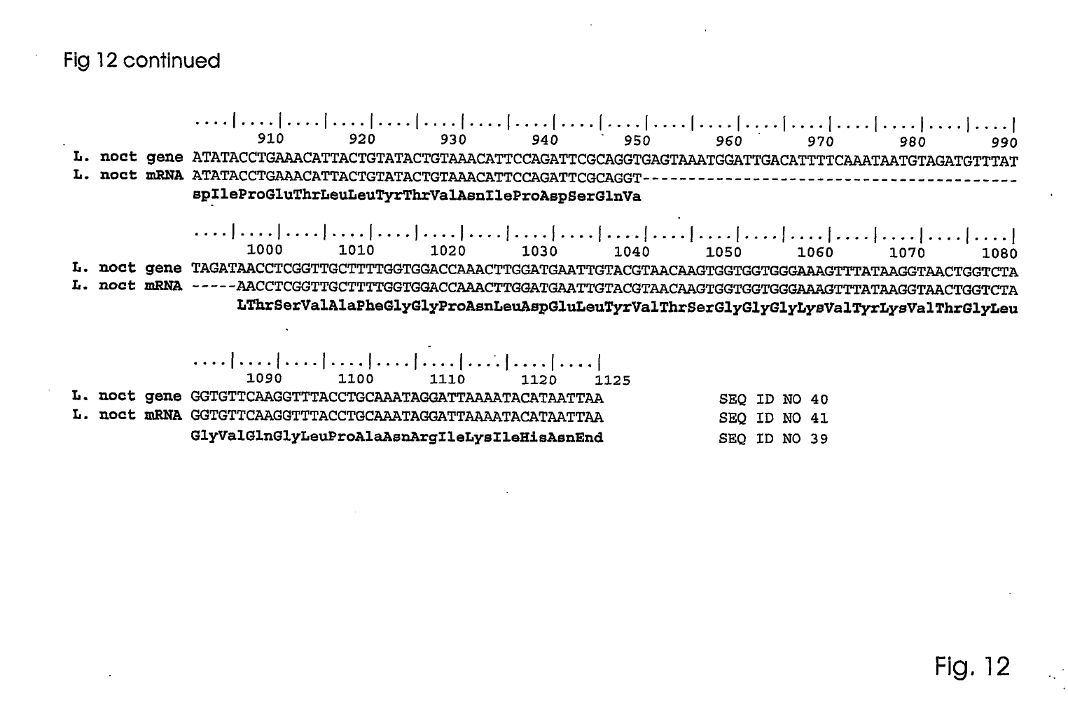

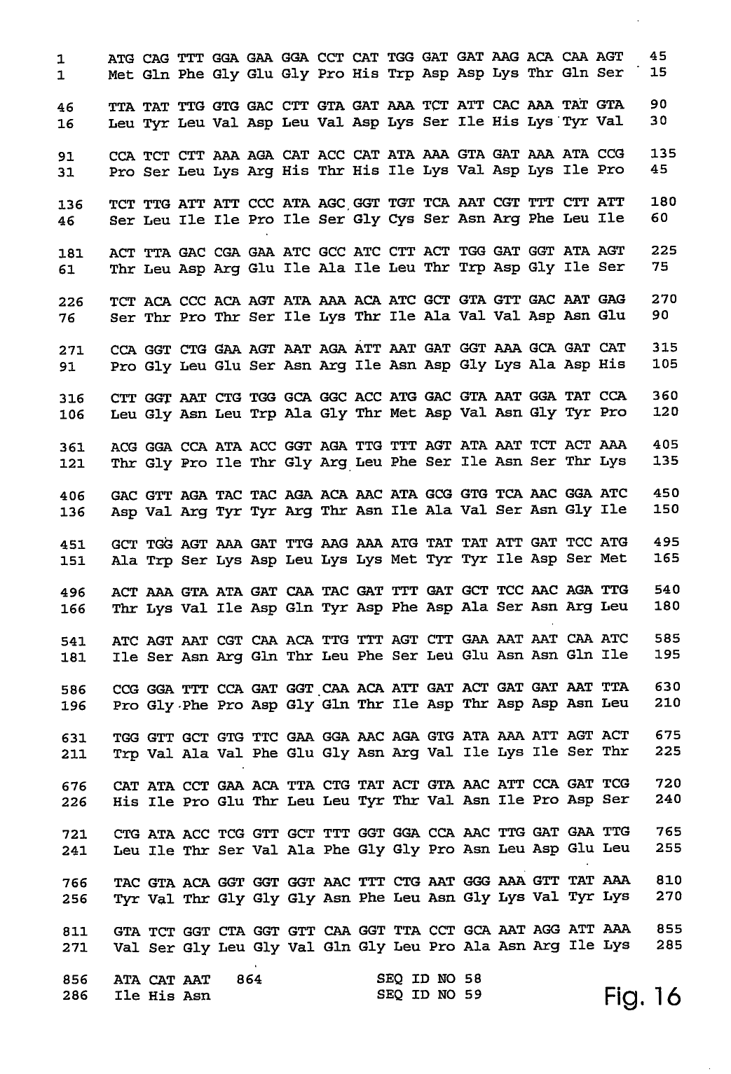

In particular, the nucleic acid encodes a luciferin recycling protein comprising SEQ ID NO 3, as shown in FIG. 10 hereinafter, or a luciferin recycling protein comprising SEQ ID NO 39, as shown in FIG. 12, or a luciferin recycling protein comprising SEQ ID NO 59, as shown in FIG. 16 or a luciferin recycling fragment or variant of any of these.

Luciferin recycling protein comprising SEQ ID NO 3, SEQ ID NO 39 or SEQ ID NO 59 are novel and as such form a further aspect of the invention, together with luciferin recycling fragments, and variants of any of these having at least 60%, more preferably at least 80%, yet more preferably at least 90% and most preferably at least 95% homology or identity.

A particular embodiment of the invention comprises a luciferin recycling protein comprising SEQ ID NO 3, as illustrated hereinafter in FIG. 10.

An alternative embodiment of the invention comprises a luciferin recycling protein comprising SEQ ID NO 39 as illustrated in FIG. 12.

A preferred embodiment of the invention comprises a luciferin recycling protein comprising SEQ ID NO 59 in FIG. 16.

Such proteins and in particular proteins of SEQ ID NO 59, are obtainable from Lampyris noctiluca and therefore allelic variants are also obtainable from this species. The sequence was cloned and partially sequenced as described in Example 1 hereinafter. The cloning process however was not straightforward, due in part to the presence of large introns, which appear to be present in native genes for luciferin recycling proteins.

Further according to the present invention, there is provided a nucleic acid that encodes a luciferin recycling protein comprising SEQ ID NO 3, a luciferin recycling protein comprising SEQ ID NO 39, or a luciferin recycling protein comprising SEQ ID NO 59, or a luciferin recycling fragment thereof, or a variant of any of these having at least 60% sequence homology or identity. In particular, the nucleic acid of the invention encodes a luciferin recycling protein comprising SEQ ID NO 3, SEQ ID NO 39 or SEQ ID NO 59.

An example of such a sequence, which encodes SEQ ID NO 3, is the genomic sequence (including introns) SEQ ID NO 4 as shown in FIG. 10. An example of a nucleic acid sequence which encodes a protein of SEQ ID NO 39 is SEQ ID NO 40 as shown in FIG. 12. A suitable cDNA sequence comprises SEQ ID NO 4 without the illustrated introns (SEQ ID NO 41) or SEQ ID NO 38 as shown in FIG. 12. In particular, the nucleic acid is of SEQ ID NO 58 as shown in FIG. 16.

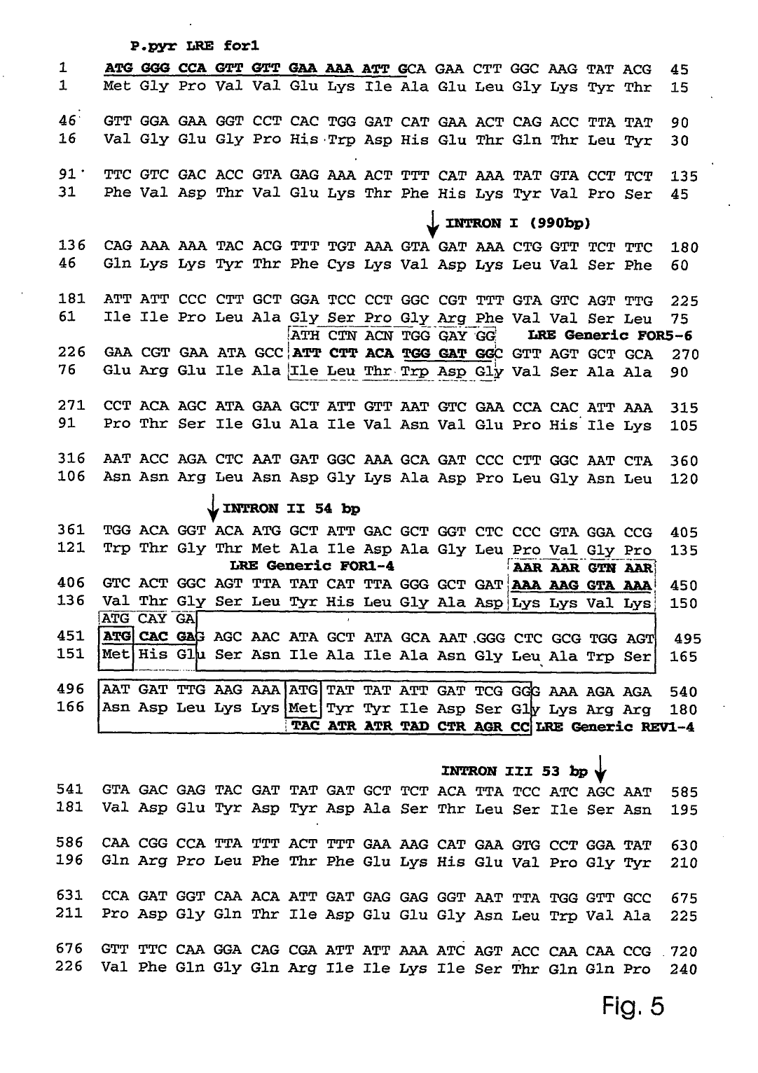

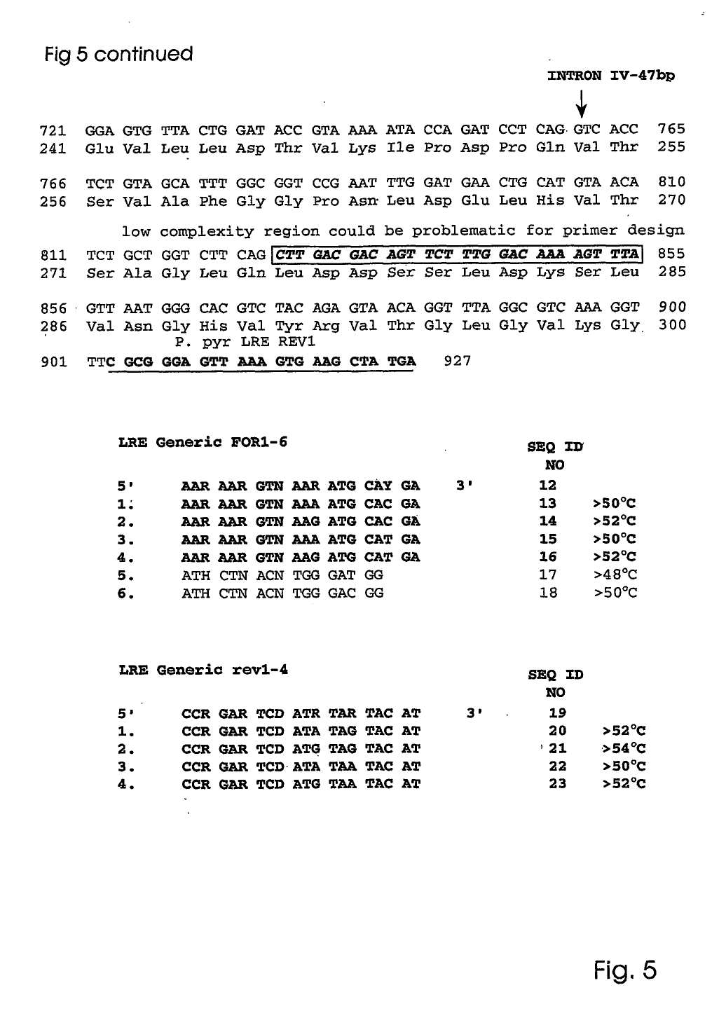

In addition to the proteins obtainable from particular species, chimeric luciferin recycling enzymes may be produced by combining fragments of enzymes from various species. In this context, the fragments suitably comprise regions encoded by individual exons found within the genomic sequences. It has been found for example, that generally the gene sequences encoding these proteins contain 5 exons, linked together by 4 introns of varying size, as illustrated hereinafter for example in FIGS. 5, 6, 7, 10, 12 and 14. Chimeric luciferin recycling proteins suitably comprise a combination of 5 individual exons, corresponding to exon I, II, III, IV and V within the sequences listed above.

Suitably, the chimeric enzyme contains at least one and preferably up to four fragments encoded by exons of a luciferin recycling gene from glowworm species such as Lampyris noctiluca. For example, the applicants have produced a luciferin recycling enzyme by splicing together exon 1 of a Photinus pyralis sequence, to a fragment of Lampyris noctiluca luciferin recycling enzyme corresponding to exons 2, 3, 4 and 5 within the gene.

An example of such an enzyme is illustrated hereinafter as SEQ ID NO 61. This sequence and luciferin recycling fragments and variants of either of these, together with nucleic acid sequences which encode these proteins, such as SEQ ID NO 60, form a further aspect of the invention.

The novel proteins of the invention may be used in the production of synthetic D-luciferin, as well as other optically active enzyme substrates. For example they may be used in the regeneration of optically active substrates of phosphatase or galactosidase enzymes.

Thus in yet a further aspect, the invention provides a method for producing an optically active enzyme substrate such as D-luciferin, which comprises contacting an oxidised form of said substrate, such as oxyluciferin, with a recycling protein comprising SEQ ID NO 3 or SEQ ID NO 39 or a luciferin recycling fragment thereof, or a variant of any of these having at least 60% homology or identity, and any other amino acid such as cysteine necessary to effect the conversion.

The reaction is suitably effected in a suitable solvent, such as an aqueous solvent, at temperatures at which the recycling enzyme is active which will vary depending upon the particular enzyme involved. For example, the reaction may be affected in the presence of a physiologically compatible buffer e.g. 25 mM-50 mM phosphate or HEPES pH 6.5-7.5 and a reaction temperature in the range 20-30° C.

The invention will now be particularly described by way of example with reference to the accompanying drawings in which:

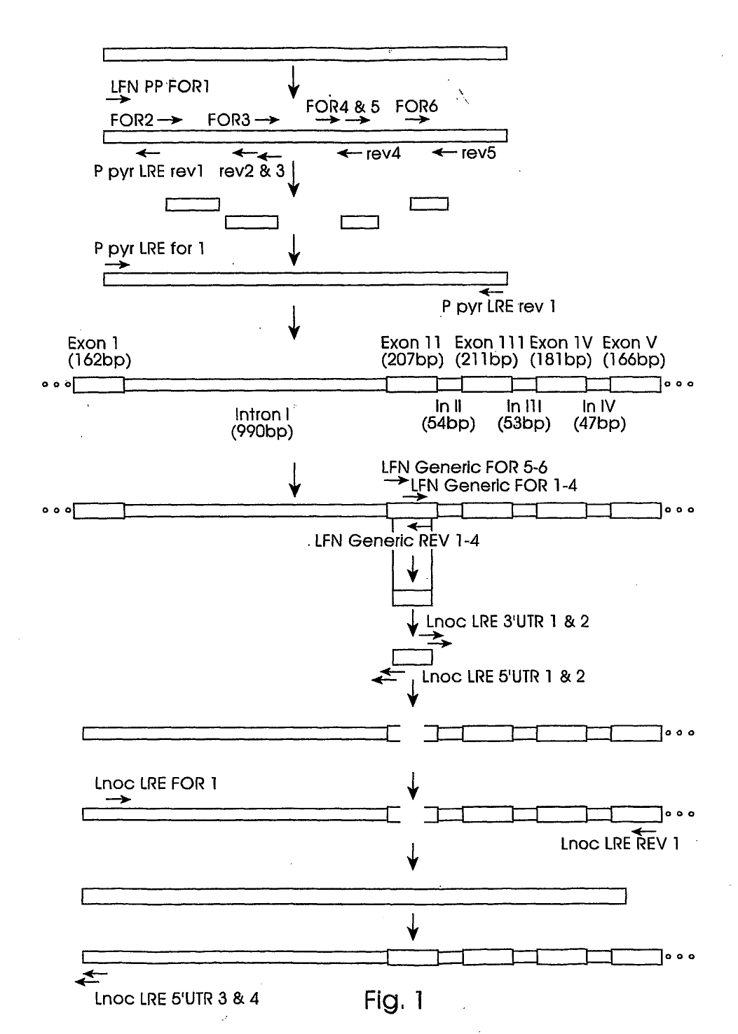

FIG. 1 shows schematically the experimental design and results obtained for the characterisation of the gene coding for a luciferin-recycling protein (enzyme) (LRE) found in L. noctiluca;

FIG. 2 shows the sequence of Photinus pyralis LRE mRNA and the corresponding protein sequence, obtained from the NCBA database (SEQ ID NO 2 and SEQ ID NO 1 respectively);

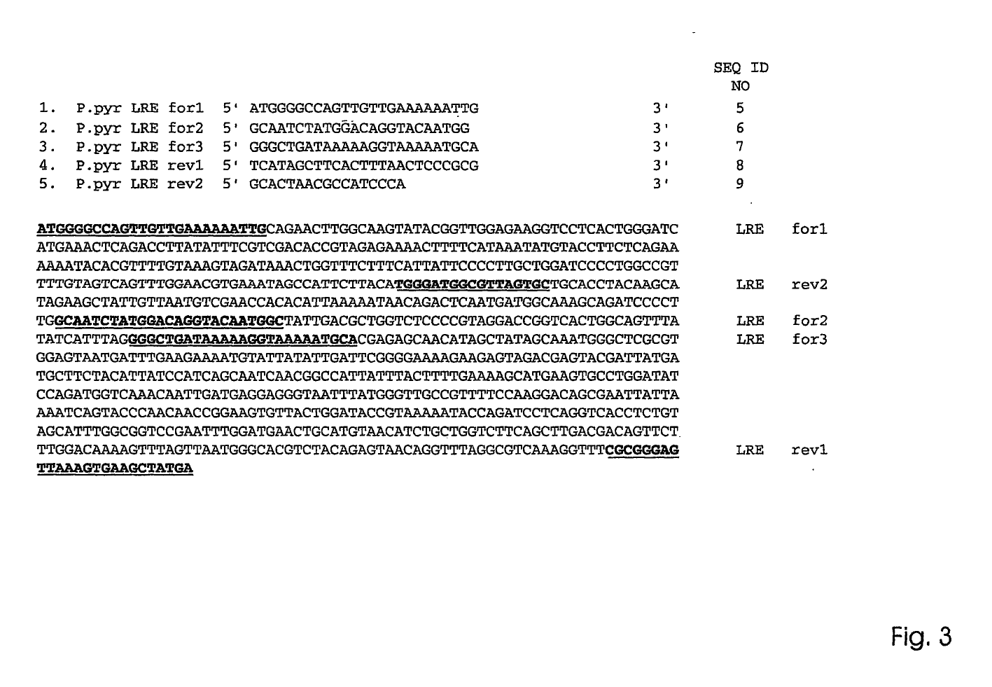

FIG. 3 shows a series of primers designed to amplify the entire gene of P. pyralis LRE;

FIG. 4 shows the complete sequence of the LRE gene from P. pyralis (SEQ ID NO 11) aligned against the mRNA sequence (SEQ ID NO 2); where exons are shown shaded;

FIG. 5 illustrates the generic primers designed to amplify small region of a L. noctiluca homologue or the P. pyralis LRE, based upon conserved amino acid sequences, and their position with respect to the amino acid sequence (SEQ ID NO 1);

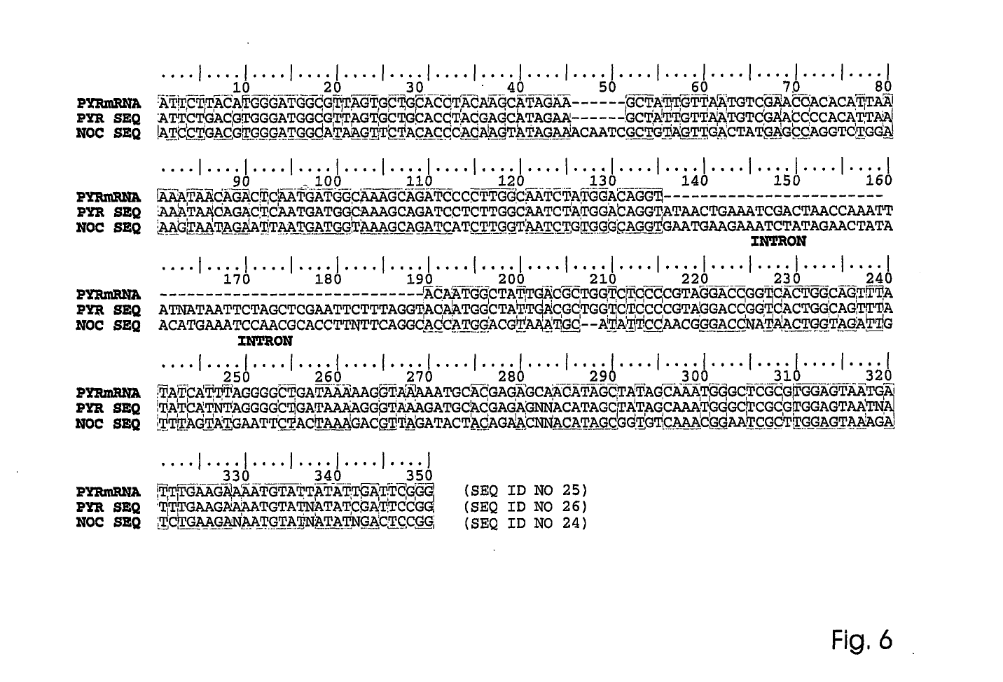

FIG. 6 shows a 350 base pair PCR product from L. noctiluca, aligned with the P.pyralis mRNA and DNA sequences;

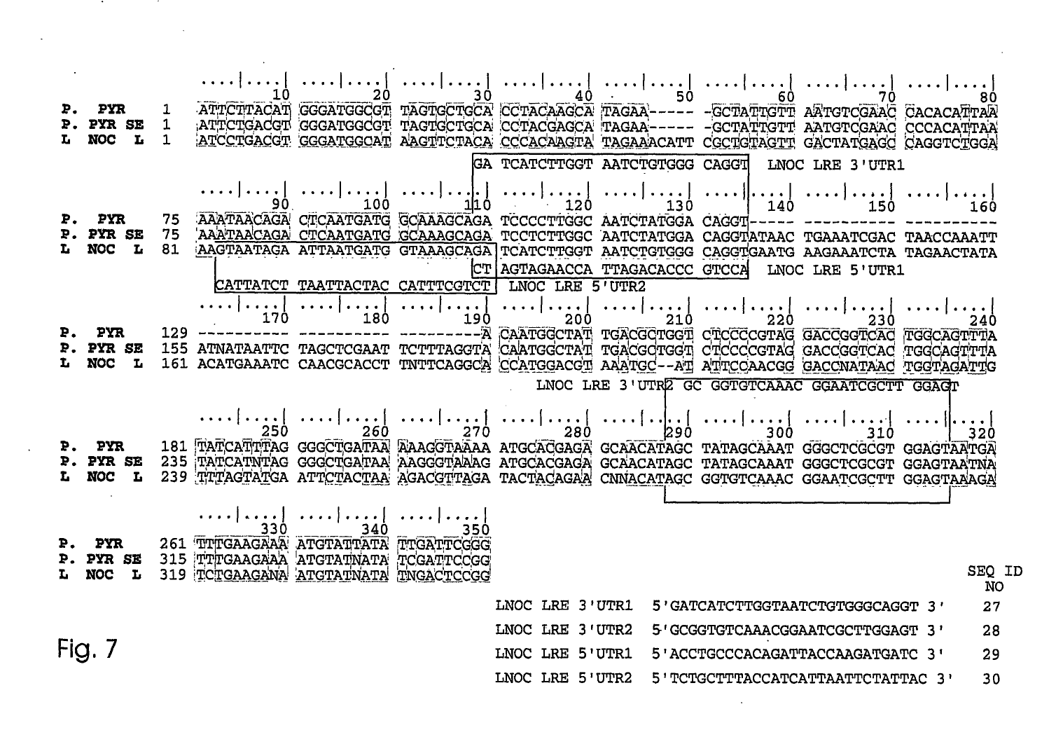

FIG. 7 shows the primers used in a 5′ and 3′ genome walking experiment;

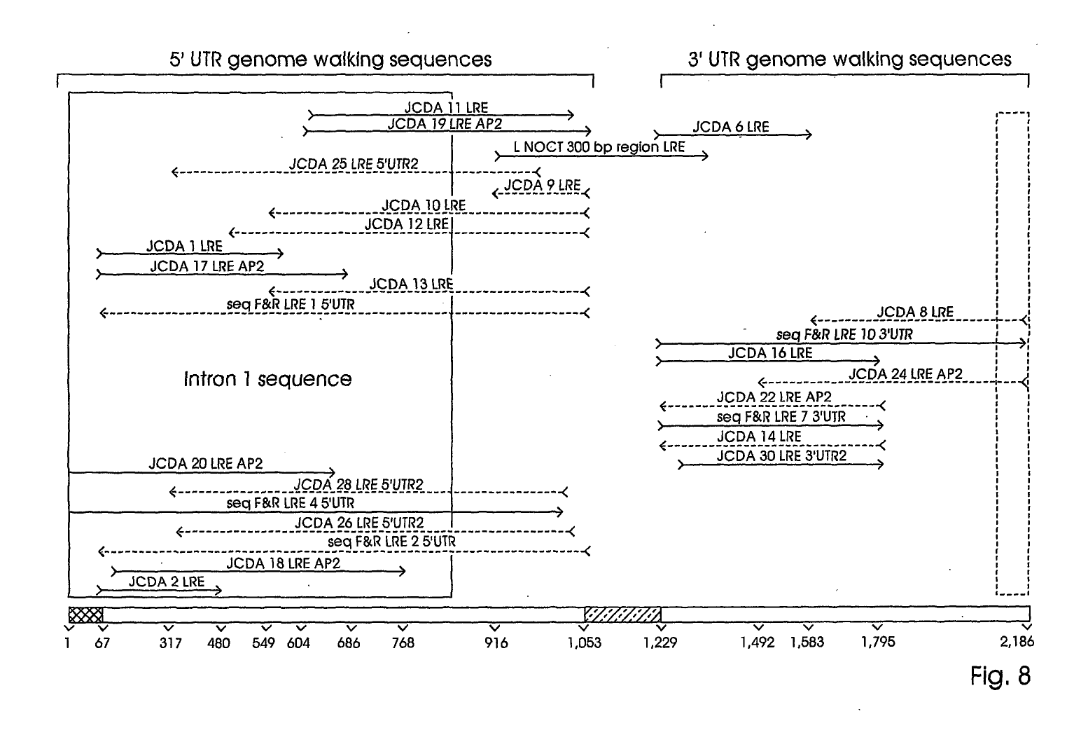

FIG. 8 illustrates the contig constructed from the genome walking experiment;

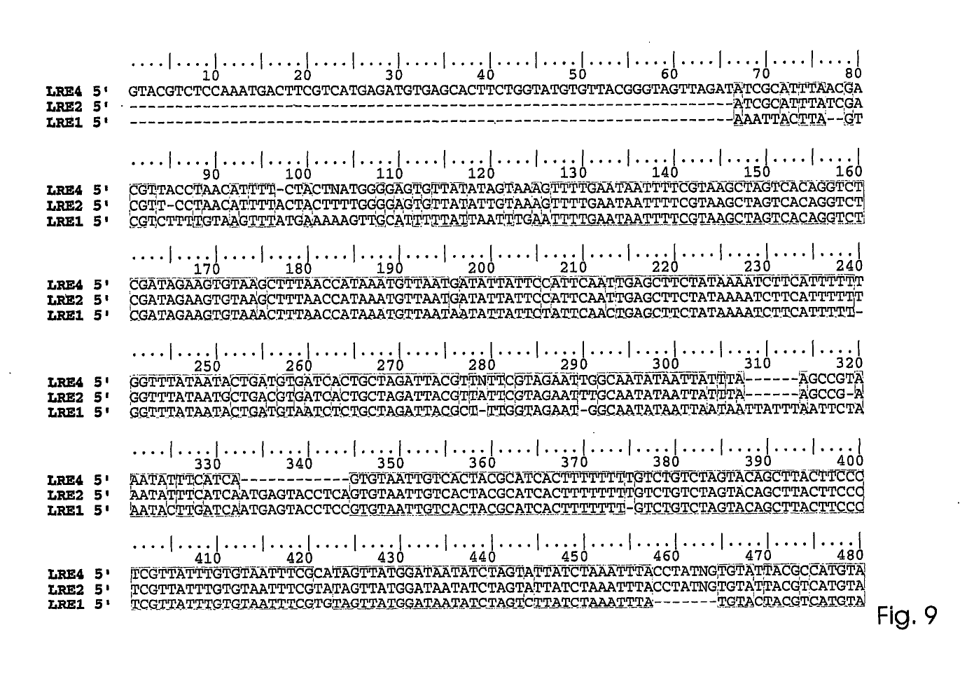

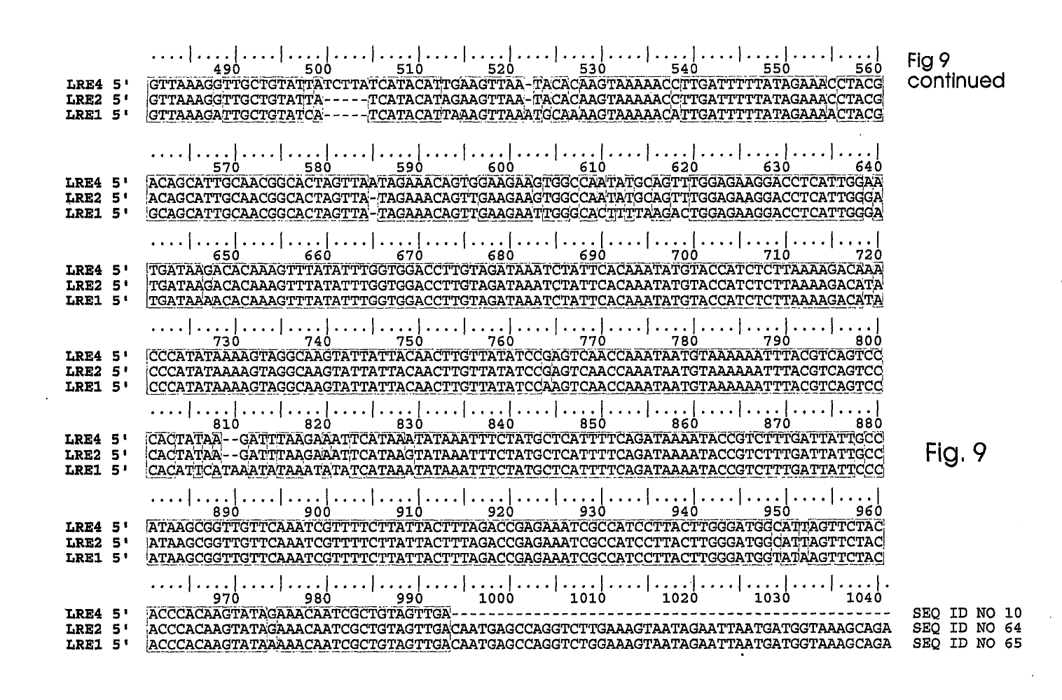

FIG. 9 shows the 5′ UTR genomic walking sequences from L noctiluca illustrating the presence of three forms of the gene;

FIG. 10 shows a protein sequence (SEQ ID NO 3) and coding nucleic acid sequence (SEQ ID NO 4) comprising the LRE of L. noctiluca;

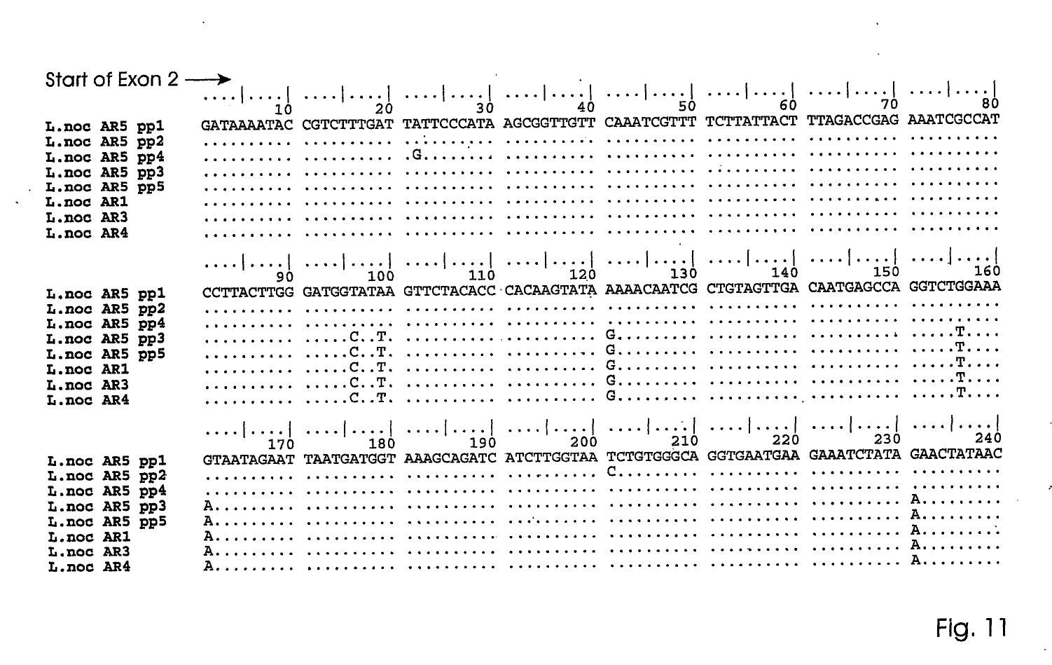

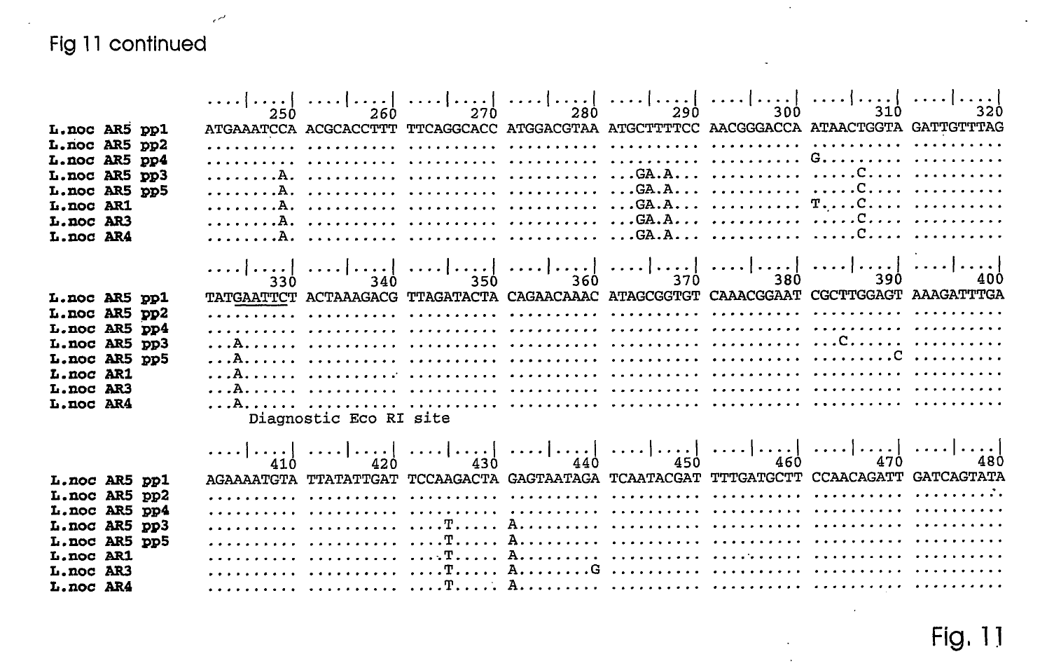

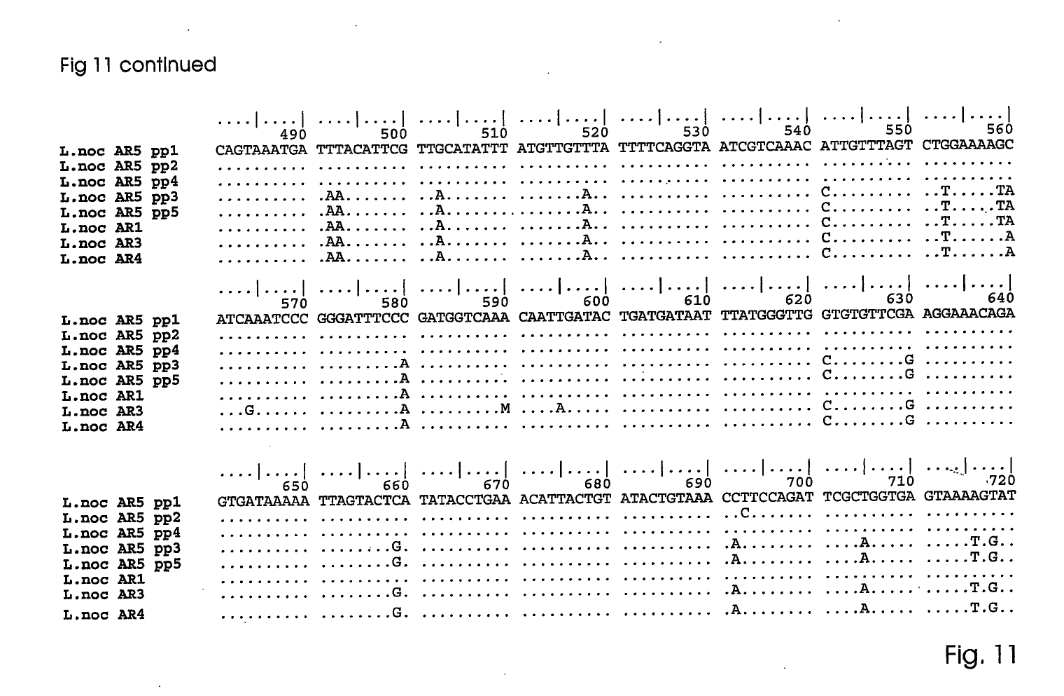

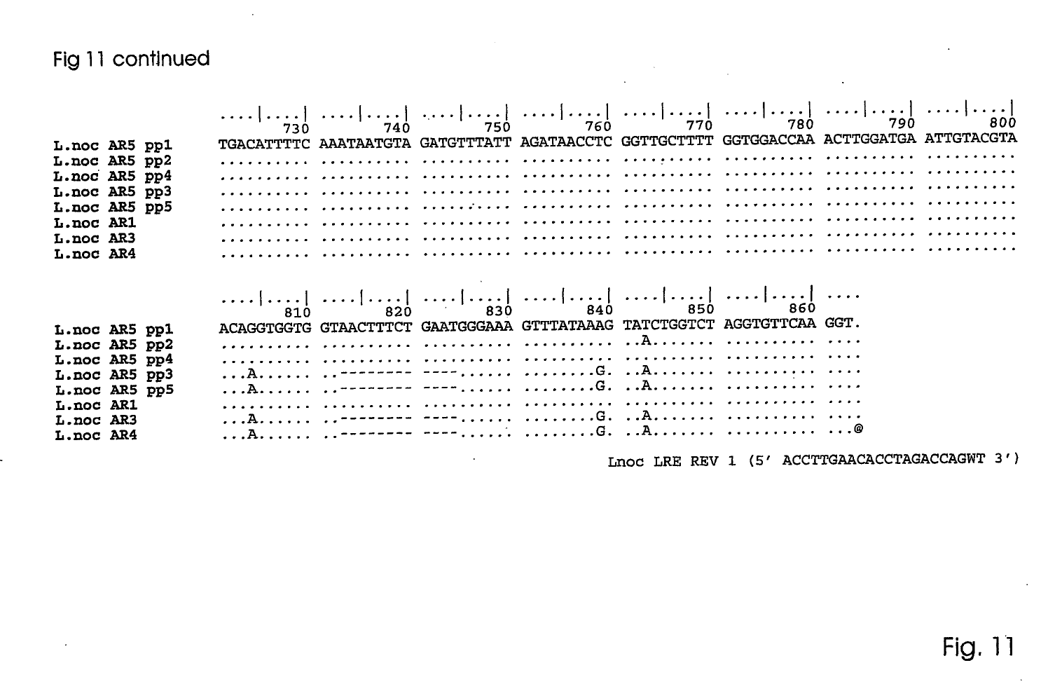

FIG. 11 shows SEQ ID NO 4 and some allelic or other variants, derived from 4 different individuals;

FIG. 12 shows a complete protein sequence (SEQ ID NO 39), the coding gene nucleic acid sequence (SEQ ID NO 40) and putative mRNA sequence (SEQ ID NO 38) comprising the LRE of L. noctiluca;

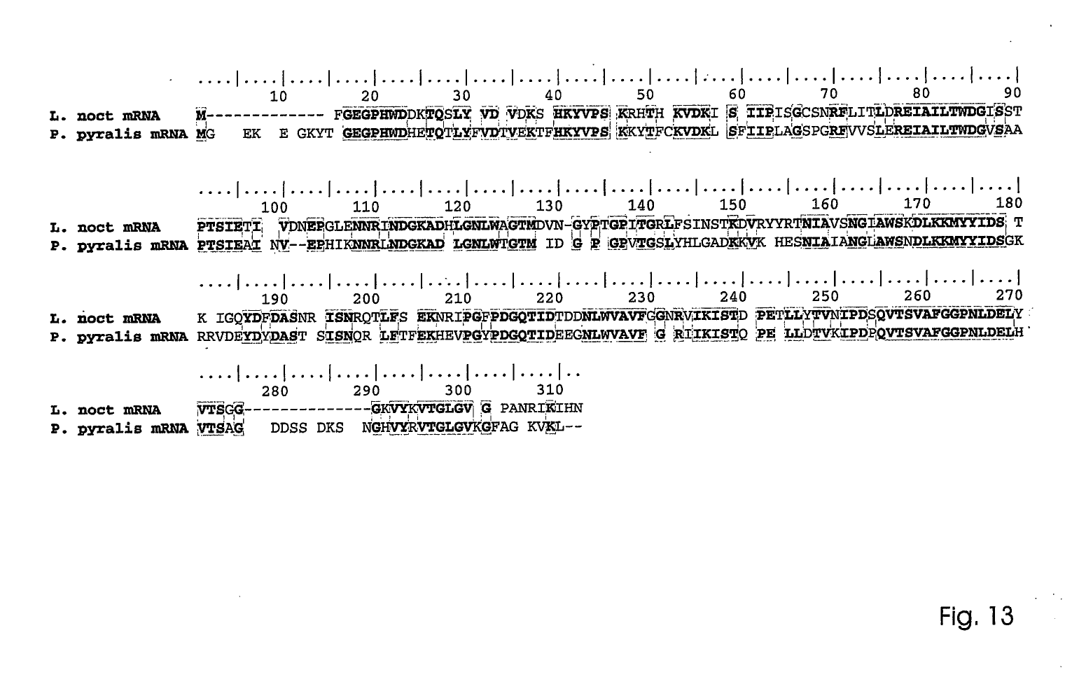

FIG. 13 shows the putative amino acid sequence derived from the complete luciferin recycling enzyme gene sequence in L.noctiluca aligned against the LRE protein sequence from Photinus pyralis (Accession number BAB60700), which displays 55.7% amino acid homology;

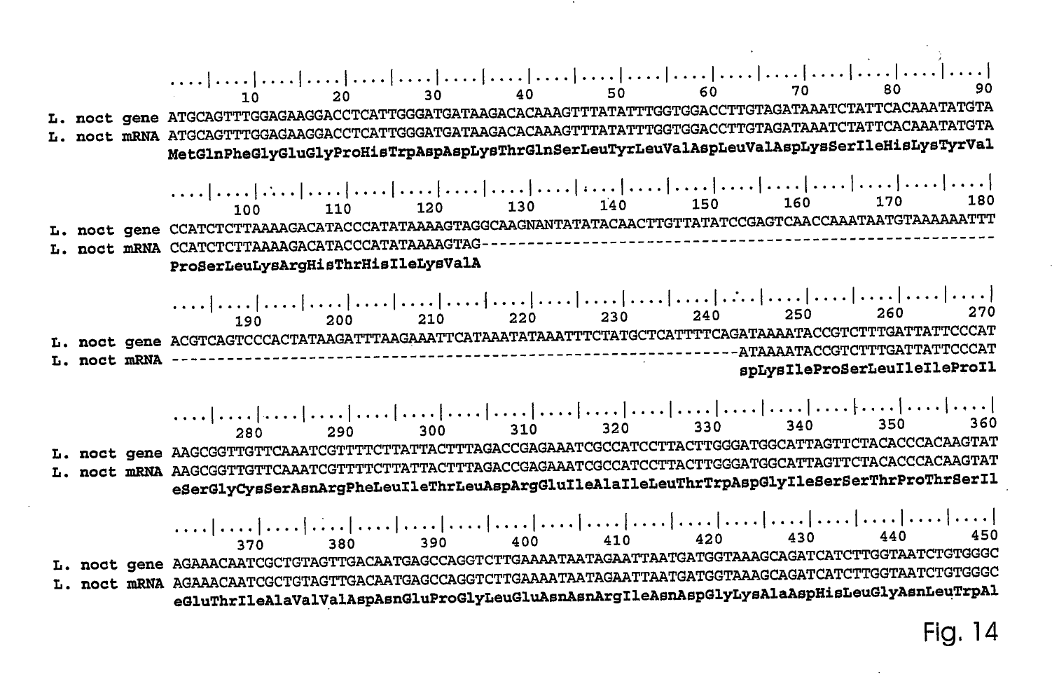

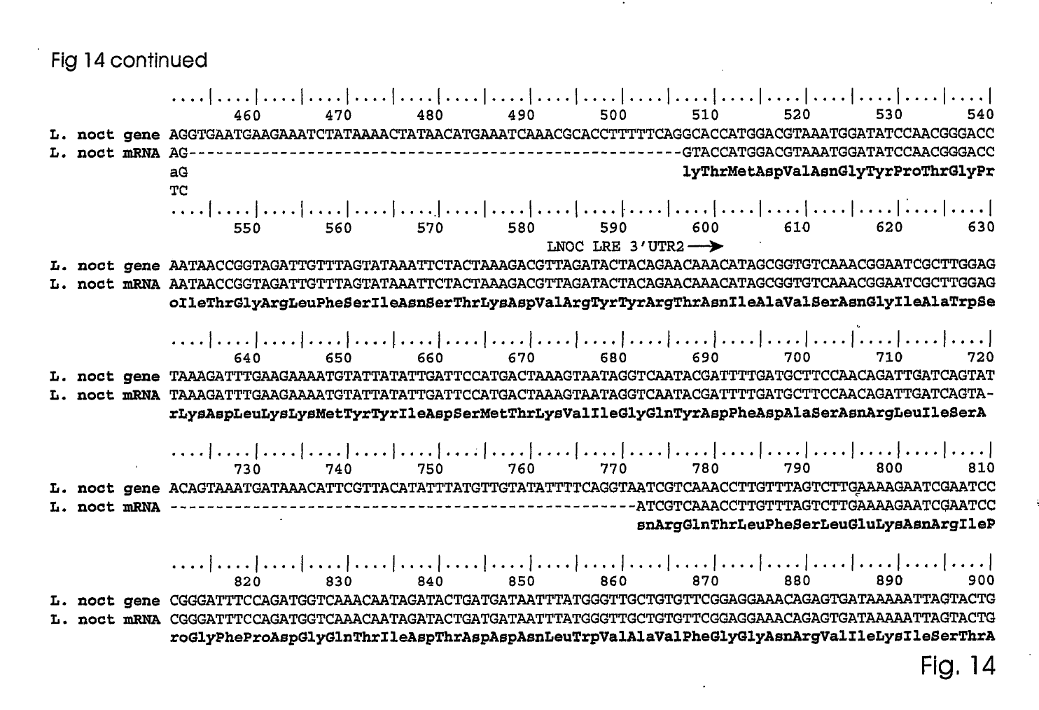

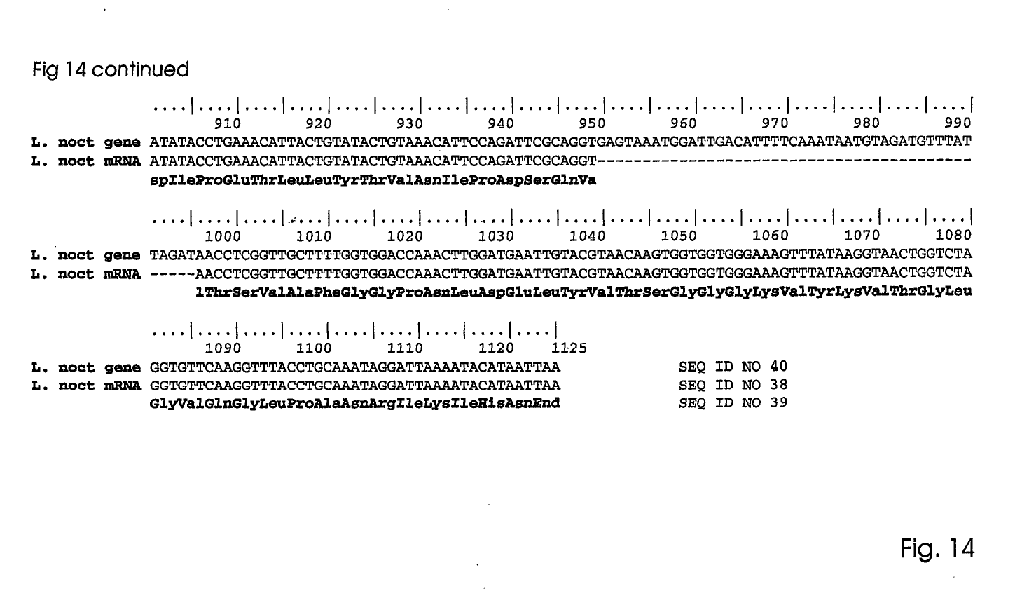

FIG. 14 is similar to FIG. 12 but showing a corrected position of intron regions within the genomic sequence (SEQ ID NO 40);

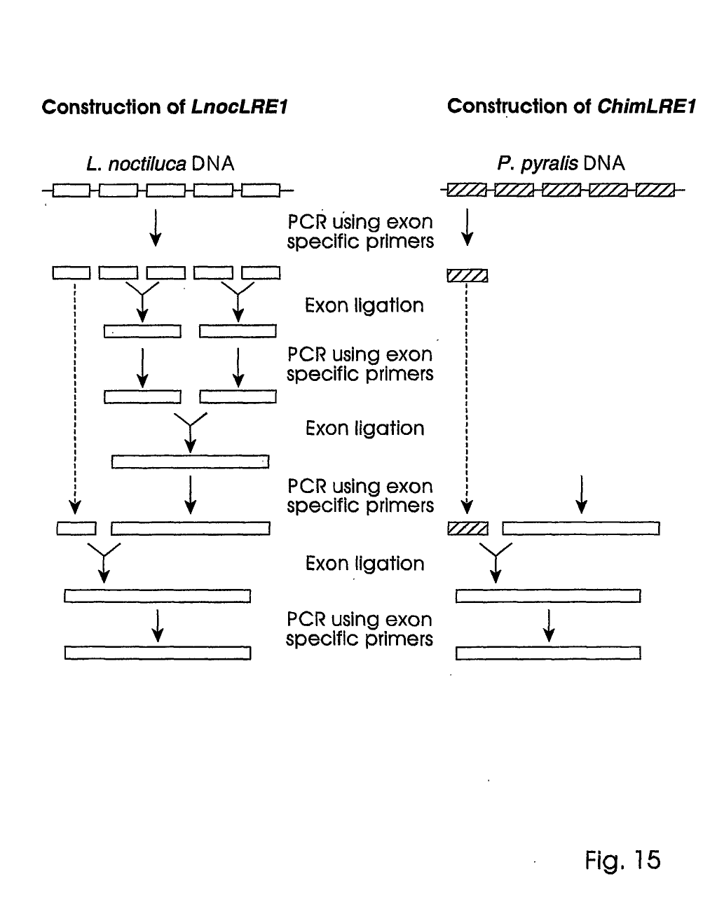

FIG. 15 is a schematic showing luciferin regenerating enzyme (LRE) cDNA construction using exon-ligation mediated PCR;

FIG. 16 shows the DNA sequence (SEQ ID NO 45) and putative amino acid sequence of L. noctiluca LRE1 (SEQ ID NO 46);

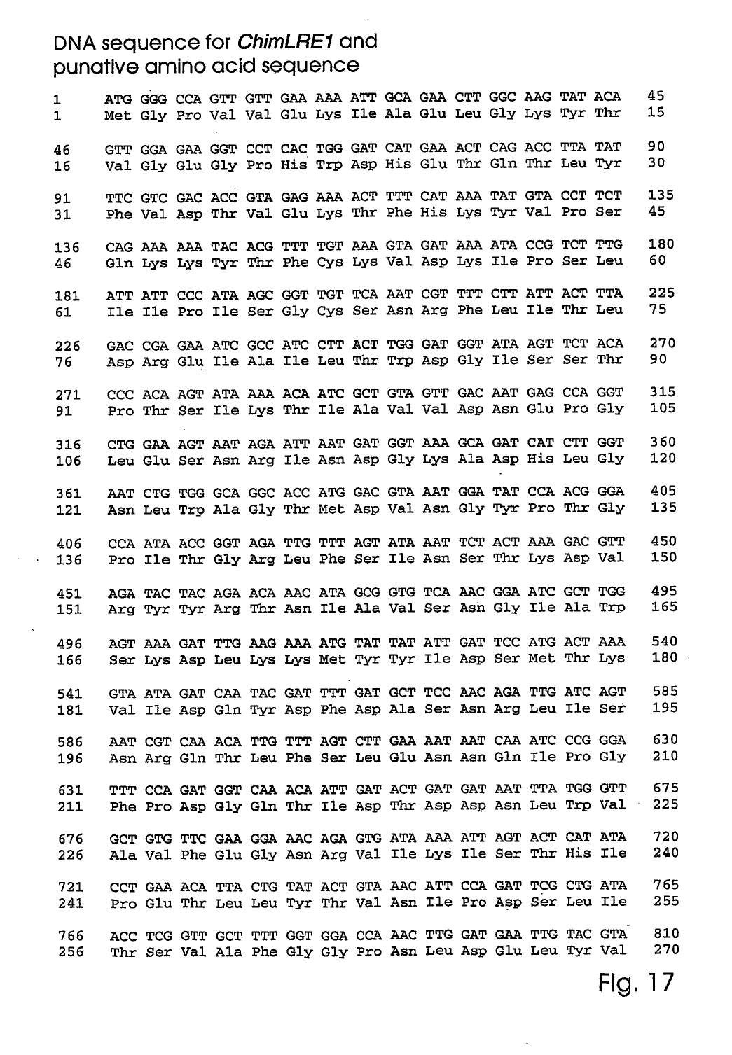

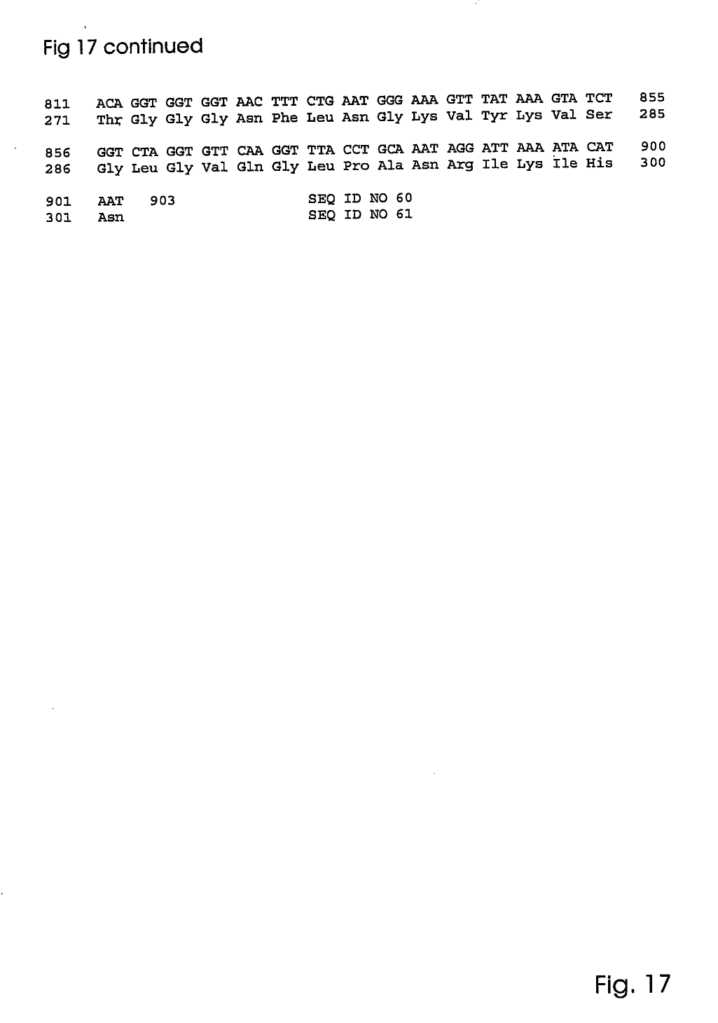

FIG. 17 shows the DNA sequence (SEQ ID NO 47) and putative amino acid sequence of L. noctiluca LRE1 (SEQ ID NO 48);

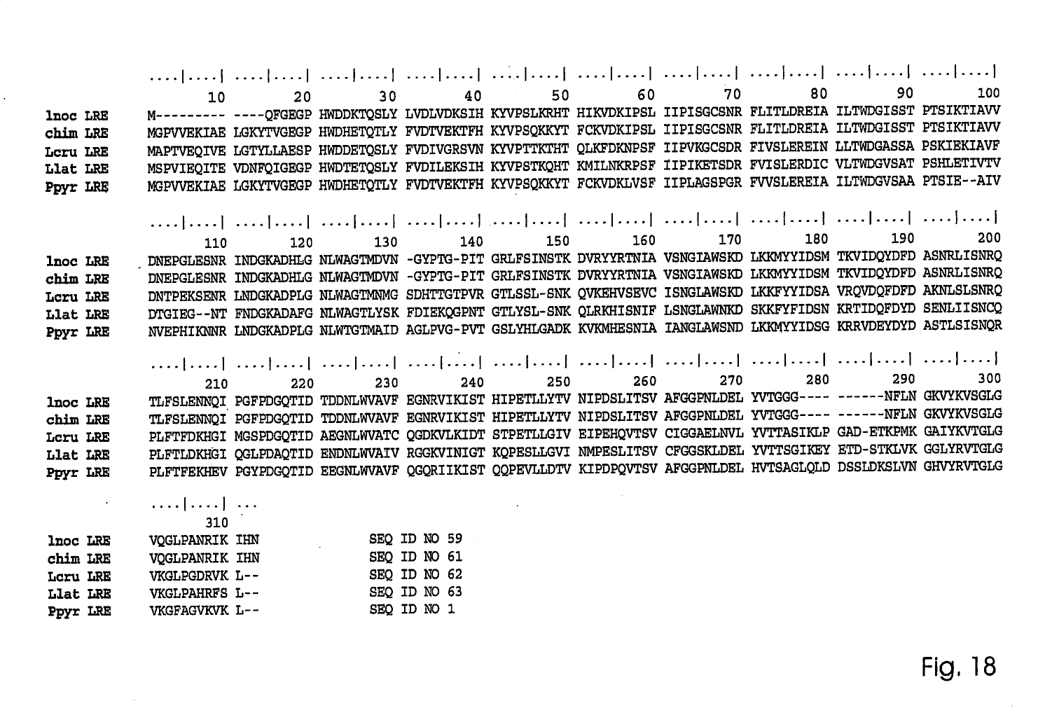

FIG. 18 shows the amino acid sequence from LnocLRE1 (lnoc LRE)(SEQ ID NO 59) and ChimLREI (chim LRE) (SEQ ID NO 61) aligned against three published LRE's; Luciola cruciata—Lcru LRE (BAB85479) (SEQ II NO 62), Luciola lateralis—Llat LRE (BAB85478) (SEQ ID NO 63) and Photinus pyralis—Ppyr LRE (BAB60700) (SEQ ID NO 1); and

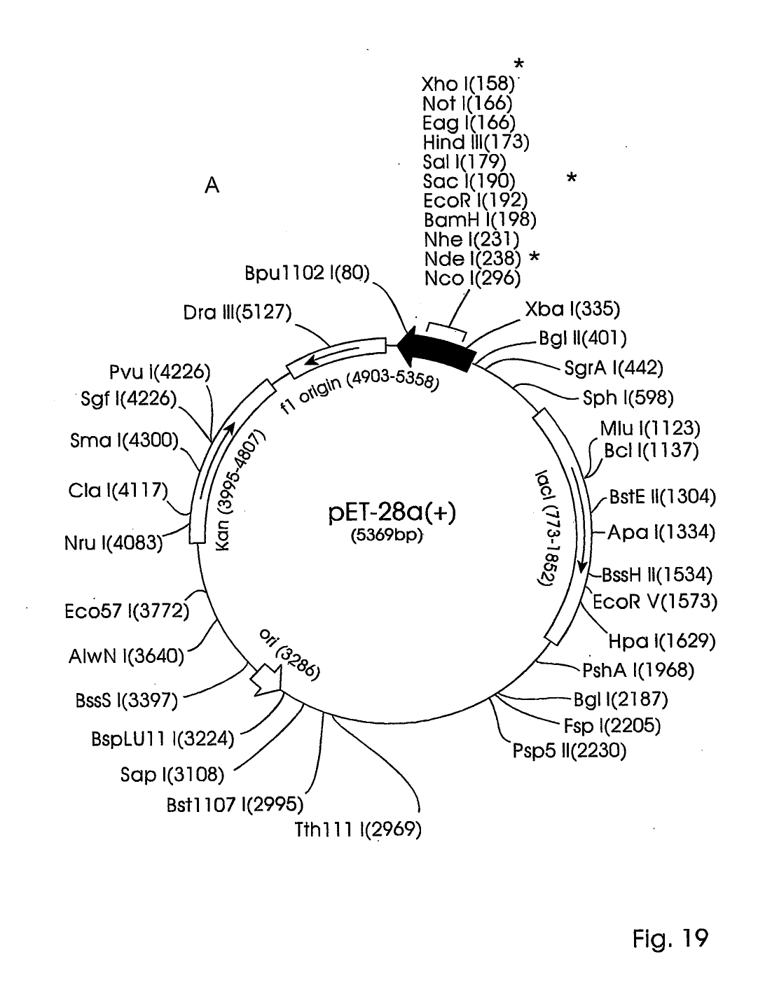

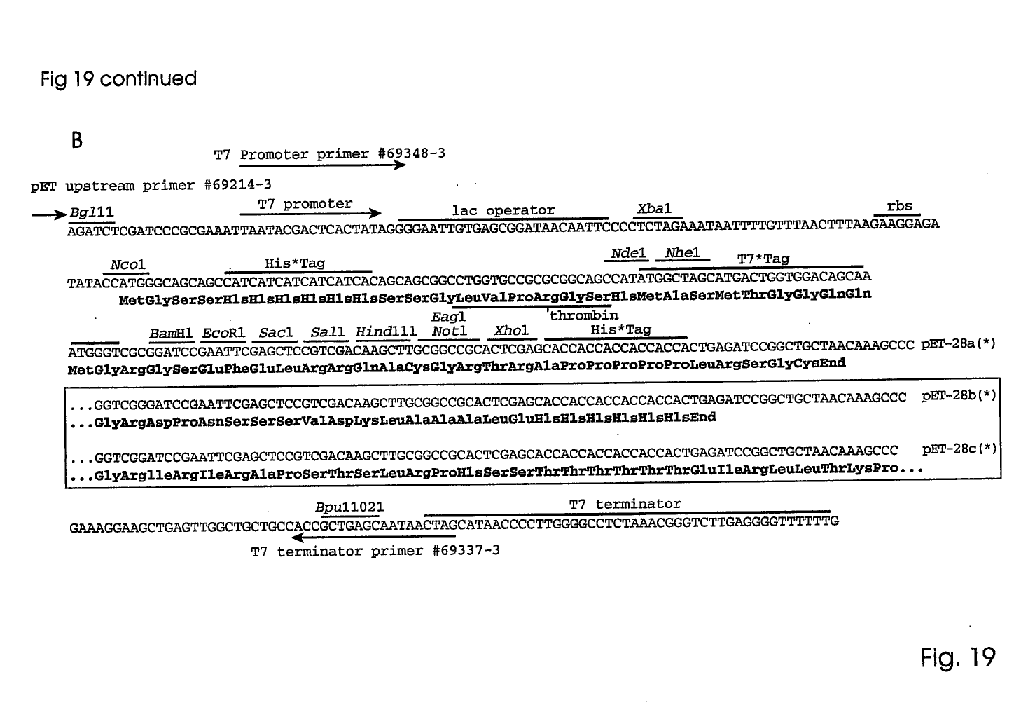

FIG. 19 illustrates the pET-28a-c(+) cloning vector (Novagen) where A. is the plasmid map for pET-28a(+), and B. shows the sequence of the pET-28a-c(+) cloning/expression region, with insertion sites marked by *.

### EXAMPLE 1

Cloning and Sequencing of an LRE Gene from Lampyris noctiluca

Lampyris noctiluca, the European glow-worm was collected from Southern England from 2000-2002. Total genomic DNA was extracting from female specimens using the High Pure PCR Template Preparation Kit (Roche). Genomic DNA was extracted in a similar manner from lyophilised Photinus pyralis specimens (Sigma).

The published P.pyralis LRE mRNA sequence (SEQ ID NO 2), obtained from GENEBANK, was used to design a range of generic primers in an attempt to amplify a homologous gene sequence from an extract of L. noctiluca. These attempts, however, were unsuccessful. Although amplification using a conventional polymerase chain reaction (PCR) yielded numerous products, these all appeared to be non-specific amplicons.

In order to overcome these difficulties, the applicants designed a set of five primers (SEQ ID NOS 5-9) to amplify the entire P. pyralis LRE gene, as illustrated in FIG. 3 hereinafter. The positioning of these primers on SEQ ID NO 2 is also shown in this Figure.

As a result of amplification using these primers, additional sequence information was generated. By combining the initial sequence with these sequences, a complete sequence contig of the P. pyralis LRE gene was generated (SEQ ID NO 11) and is shown in FIG. 4. In this Figure, the complete gene sequence is aligned against the mRNA sequence (SEQ ID NO 2). The exons were characterised and are shown shaded.

Generic primers were designed to amplify a small region of a L. noctiluca homologue of the P. pyralis LRE, based upon conserved amino acid sequences. These consisted of 6 forward primers (SEQ ID NOS 12-18) and 5 reverse primers (SEQ ID NOS 19-23). The sequence of the primers and their position with respect to the amino acid sequence (SEQ ID NO 1) is illustrated in FIG. 5.

Using the LRE generic primers FOR5 (SEQ ID NO 17) and LRE generic REV 3 (SEQ ID NO 22), a 350 base pair PCR product was amplified from L. noctiluca. This was sequenced (SEQ ID NO 24) and shown to have homology with sequences in the P.pyralis LRE mRNA and genomic sequence (SEQ ID NOs 25 and 26 respectively) as illustrated in FIG. 6. This gave assurance that there was indeed a homologue LRE in L.noctiluca.

With this information, 4 primers (SEQ ID NOs 27-30) were designed with a view to carrying out 5′ and 3′ genome walking along the L. noctiluca LRE gene. The sequence of these primers and the location on SEQ ID-NOs 24-26 is illustrated in FIG. 7.

As a result of the 5′ and 3′ genome walking exercise with these primers, six clones were obtained from both the 5′ and 3′ ends and these were sequenced both forward and reverse. The resulting contig included the complete sequence downstream of the original 350 bp sequence. The sequence at the 5′ end has no homology to any regions of exon/intron I in P.pyralis. The contig, illustrated in FIG. 8, of all the walking sequences produced more than two alleles in the alignment, suggesting that there might be more than one locus for this gene in L. noctiluca.

This is further illustrated in FIG. 9 which shows the 5′UTR genomic walking sequences from L. noctiluca. Three different allelic forms of the gene are shown in one individual (SEQ ID NOS 10, 64 and 65), suggesting the presence of two or more copies of the gene. In this Figure, exon II begins at the arginine and position 855 in the illustrated sequence.

The 3′UTR genomic walking sequences (not shown) suggested that there were two different forms of the gene in one individual.

A sequence of a major part of the L. noctiluca LRE protein and the coding sequence as derived from this exercise are illustrated in FIG. 10 as SEQ ID NO 3 and SEQ ID NO 4 respectively.

Some allelic variants from four different individuals are shown in FIG. 11 as SEQ ID NOS 31-37.

It was surprisingly found that intron I of L. noctiluca was considerably smaller than intron I from P. pyralis. A complete read out beyond intron I and exon I in L. noctiluca was obtained. The complete gene sequence for the luciferin recycling protein from Lampyris noctiluca, the putative mRNA and protein sequence are shown in FIG. 12 as SEQ ID NOS 40, 41 and 39 respectively.

### Example 2

Preparation of Luciferin Recycling Proteins

Based upon the LRE gene sequence from Lampyris noctiluca (FIG. 14) five sets of primers, NOC LRE EX1-5 were designed to amplify each exon individually (FIG. 15, Table 1).

TABLE 1

List of-oligonucleotides and sequences. Underlined
bases denote restriction enzyme sites.

| | | SEQ ID |
|---|---|---|
| Oligonucleotide | Sequence 5′-3′ | NO |
| NOC LRE EX1 for | ATGCAGTTTGGAGAAGGACCTCATTGGG | 42 |
| NOC LRE EX1 rev | CTACTTTTATATGGGTATGTCTTTTAAG | 43 |
| NOC LRE EX2 for | ATAAAATACCGTCTTTGATTATT | 44 |
| NOC LRE EX2 rev | CTGCCCACAGATTACCAAGATGATCTG | 45 |
| NOC LRE EX3 for | GCACCATGGACGTAAATG | 46 |
| NOC LRE EX3 rev | TGATCAATCTGTTGGAAGCATCA | 47 |
| NOC LRE EX4 for | GTAATCGTCAAACATTGTTTAGTC | 48 |
| NOC LRE EX4 rev | CAGCGAATCTGGAAKGTTTACAG | 49 |
| NOC LRE EX5 for | ATAACCTCGGTTGCTTTTGGTGACC | 50 |
| NOC LRE EX5 rev | ATTATRTATTTTWATCCTATTTGCAG | 51 |
| (1097) | | |
| PYR LRE EX1 for | ATGGGGCCAGTTGTTGAAAAAATTGCAG | 52 |
| PYR LRE EX1 rev | TCATAGCTTCACTTTAACTCCCGC | 53 |
| NOC LRE Nco I | GAGCTCCCATGGGCCAGTTTGGAGAAGGACCTCAT | 54 |
| START | TGGG | |
| NOC LRE Xho I | GAGCTCTCGAGATTATRTATTTTWATCCTATTTGC | 55 |
| END1 | AG | |
| NOC LRE Xho I | GAGCTCTCGAGTTAATTATRTATTTTWATCCTATT | 56 |
| END2 | TGCAG | |
| PYR LRE Nco I | GAGCTCCCATGGGGCCAGTTGTTGAAAAAATTGC | 57 |
| START | | |

PCR was carried out using the proof reading PFU polymerase (Promega) in order to minimise the chance of a single deoxyadenosine residue being added to the 3′ ends of the amplified fragments.

PCR products were separated on a 2% agarose gel, excised from the gel and extracted using agarose clean up columns (AB Gene). In the first ligation round, 2 exons pairs were ligated together. Ligation reactions were carried out at 16° C. overnight. 0.2 μl of the ligation reaction was used in the second round of PCR. For each ligated pair of exons one forward and one reverse primer were used to amplify a contiguous product (FIG. 15). Electrophoresis, gel excision and clean up was carried out as described above. In the second round of ligation, the two pairs of exon PCR products were ligated together. PCR amplification generated contiguous products that were subsequently used in a final ligation with the remaining exon.

The final PCR product containing all five exons ligated sequentially was cloned using the pGEM®-T Easy Vector System (Promega) and sequenced using a dye termination kit (Beckman) to confirm a continuous open reading frame and thus generating a complete transcript—LnocLRE1 (FIG. 16). Due to the apparent differences in exon 1 of the LRE between P. pyralis and L. noctiluca, it was decided to amplify exon 1 from P pyralis, using P. pyralis specific primers (Table 1), and to splice this onto the exons 2-5 of L. noctiluca to produce a chimaeric LRE—ChimLRE1 (FIG. 17).

Sequence Characteristics

Two transcripts generated from exon ligation mediated PCR, LnocLRE1 and ChimLRE1 are 864 bp and 903 bp in length respectively. Both sequences (SEQ ID NOS 58 and 60 respectively) are shown, with putative amino acid sequences (SEQ ID NOS 59 and 61 respectively) in FIGS. 16 and 17. These amino acid sequences are shown aligned with other LRE's previously reported (FIG. 18) and amino acid percentage identities are shown in table 2.

TABLE 2
Percentage Amino acid indentity between LnocLRE1 and
ChimLREI against three published LRE's; Luciola cruciata - Lcru
LRE (BAB85479), Luciola laterlais - Llat LRE (BAB85478) and
Photinus pyralis - Ppyr LRE (BAB60700).

| | ChimLREI | Lcru LRE | Llat LRE | Ppyr LRE |
|---|---|---|---|---|
| LnocLRE1 | 90.6 | 51.5 | 50.6 | 54.1 |
| ChimLREI | | 53.2 | 51.6 | 63.1 |
| Lcru LRE | | | 57.2 | 56.2 |
| Llat LRE | | | | 50.8 |

Transformation into stable and expression strains of E. coli Both LRE sequences can be transferred to a suitable expression vector by using restriction enzyme site containing primers for PCR amplification to enable correct orientation and expression in the chosen vector. Primers were designed with a Nco I and a Xho I site (Table 1). Restriction enzyme digestion of PCR products will result in the conversion of sticky end cloning sites, a Nco I site at the 5′ end and an Xho I site at the 3′ end.

Particular vectors which may be used for expression of LRE sequences are pET-28a (FIG. 19) and 16b cloning vectors (Novagen). The former will produce proteins with a C-terminal His tag. The latter provides for native protein production (see cloning strategy above).

Target genes cloned in pET plasmids are under the control of a strong T7 promoter and require expression in a host containing the T7 RNA polymerase gene. This is provided by the DE3 lysogen of the BL21 (DE3) host strains. However, as pET recombinants can be unstable in the expression strains (DE3), plasmids are maintained in a stable E. coli strain (XL1-Blue). Recombinant plasmids can then be transformed into the expression strain BL21(DE3)pLysS prior to induction. The expression strain pLysS provides for high-stringency expression.

2 μl of each ligation reaction is added to 100 μl of competent cells (XL1-Blue) and subjected to 45 second heat shock at 42° C. Following 2 minutes incubation on ice 0.9 ml of SOC is added and the sample incubated, with shaking at 37° C., for 1 hr.

Recombinants are selected on LB plates containing 50 μg/ml ampicillin (for pET) and 34 μg/ml chloramphenicol (for pLysS).

Recombinants may be sequenced prior to transformation of 25 ng into the expression strain BL21(DE3)pLysS.

Over-Expression

An overnight culture (3 ml) of each construct (in LB containing ampicillin, 50 μg/ml, and chloramphenicol, 34 μg/ml, is used to inoculate a 1 L culture. Prior to induction cultures are split to provide a control (uninduced sample) and a test (induced sample). All test samples are induced at an optical density (A₆₀₀) of between 0.45-0.6 with 1 mM isopropyl-β-D-thiogalactoside (IPTG), for 3-5 hours.

To check for protein expression 1 ml samples of both uninduced and induced cultures were pelleted.

To each pellet, 100-200 μl of 1×SDS solubilising buffer (SB) is added and the samples boiled for 5 minutes prior to sodium dodecyl sulfate-polyacrylamide gel analysis (SDS-PAGE, Laemmli, U. K. (1970). Cleavage of structural proteins during the assembly of the head of bacteriophage T4. Nature 227(259): 680-85).

To liquid fractions of any protein prep an equal volume of 2×SB is added prior to denaturing and SDS-PAGE.

Solubilising buffer:

| | 1X | 2X |
|---|---|---|
| 10% SDS | 100 | 200 |
| 1M Tris | 50 | 100 |
| 2-mercaptoethanol | 50 | 100 |
| PMSF | 20 | 40 |
| EDTA | 10 | 20 |
| Glycerol | 100 | 200 |
| DH₂0 | 670 | 340 |
| | 1000 μl | 1000 μl |

Add about 50 μl 10 mg/ml bromophenol blue.

SDS-PAGE gels are prepared using a 10% resolving gel and a 4% stacking gel. They are run for approximately 1 hour at 25-30 mA/gel in Tris/glycine/SDS buffer [0.025M Tris, 0.192M glycine, 0.1% SDS (ICN)]. Proteins are visualised by incubating, with shaking, in coomassie blue (1 hr) and destained until protein bands are seen clearly using destain (see below).

10% Resolving gel (makes 4-5):

| | |
|---|---|
| dH₂O | 16.2 ml |
| 1.5M Tris; 0.384% SDS (Protogel buffer | 10 ml |
| from National Diagnostics) | |
| 10% SDS | 450 μl |
| Ultra Pure Protogel (30% w/v acrylamide | 13.3 ml |
| from National Diagnostics) | |
| 10% APS (made fresh each time) | 400 μl |
| Temed | 40 μl |

4% stacking gel (makes 4-5):

| | |
|---|---|
| dH₂O | 12.2 ml |
| 0.5M Tris; 0.4% SDS (Protogel buffer from | 5 ml |
| National Diagnostics) | |
| 10% SDS | 200 μl |
| Ultra Pure Protogel (30% w/v acrylamide | 2.7 ml |
| from National Diagnostics) | |
| 10% APS (make fresh each time) | 200 μl |
| Temed | 40 μl |

Coomassie Brilliant Blue

| | |
|---|---|
| Coomassie brilliant blue R-250 | 1 g |
| Methanol | 450 ml |
| Water | 450 ml |
| Acetic acid | 100 ml |

Destain

| | |
|---|---|
| Isopropanol | 125 ml |
| Acetic acid | 50 ml |
| Glycerol | 15 ml |
| Water | 310 ml |

Protein Purification

Induced cultures (500 ml) are collected and resuspended in 20 ml buffer. Soluble crude extracts (clarified samples) are prepared by sonication (10 cycles of 25 sec on, 20 sec off) and collection of supernatants following centrifugation (14000 rpm, 20 min). His-tagged proteins are purified using the TALON affinity resin according to the manufacturer's instructions (BD biosciences). TELON resin utilises immobilised cobalt 2+ and provides enhanced selectivity for polyhistidine-tagged proteins.

Purified proteins can be visualised using SDS-PAGE and quantified according to the Bradford assay (Bradford, M. M. (1976) A rapid and sensitive method for the quantification of microgram quantities of protein utilizing the principle of protein-dye binding. Anal. Biochem. 72: 248-54).

### Example 3

In-Vivo Assay using Luciferase Recycling Enzyme

Whole cells are transformed using conventional methods, so that they express an LRE and firefly luciferase activities. These cells are incubated in a low pH buffer (max pH 5.0) containing 0.6 mM D-luciferin, for 0.5 to 10 min (depending on cell type).

The cells are then removed from the low pH buffer, washed, and resuspended in a neutral buffer. Alternatively, the low pH buffer is neutralised using a suitable alkali. Bioluminescence emitted from the cells can then be detected and measured using a luminometer or other light-detecting instrument. This provides an in vivo assay for measuring ATP intracellularly.

### Example 4

In-Vitro Assay using Luciferase Recycling Enzyme

A sample containing ATP to be assayed will be added to a reaction mixture containing 25 mM Tricine-NaOH pH 7.8, 4.0 mM MgSO4, 0.1-10 μg firefly luciferase and 0.1-20 μg purified recombinant LRE according to the invention, or 0.1-40 μg purified recombinant luciferase-LRE fusion protein also according to the invention, 0.5-5 mM D-cysteine and 250 μM D-luciferin. Light output from the reaction will be detected and measured using a luminometer or other light detecting instuments, as a measure of ATP content of the sample.

All references mentioned in the above specification are herein incorporated by reference. Other modifications of the present invention will be apparent to those skilled in the art without departing from the scope and spirit of the invention. Although the invention has been described in connection with the specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

1. A method for measuring intracellular ATP or gene expression, said method comprising transforming a cell with a construct which encodes a first luciferase and a luciferin recycling protein, introducing luciferin into said cell, and monitoring bioluminescent signals from said cell.

2. The method of claim 1, wherein the method is used to monitor intracellular ATP levels.

3. The method of claim 1, wherein the method is used to monitor luciferase expression.

4. The method of claim 1 wherein the cell is contacted with luciferin and the pH is reduced to less than 5, thereafter, the pH is restored to physiological pH conditions to monitor bioluminescent signals.

5. The method according to claim 1, wherein the cell is further transformed such that it expresses an L-cysteine racemase.

6. The method of claim 1, wherein the cell is capable of expressing a second luciferase which has a different Kₘ value and a different wavelength output than said first luciferase.

7. A composition comprising a DNA construct comprising a nucleic acid sequence which encodes a luciferase enzyme, and a nucleic acid sequence which encodes a luciferin recycling protein.

8. The composition of claim 7 wherein the nucleic acid sequences are linked to express a fusion protein comprising a luciferase and a luciferin recycling protein.

9. The composition of claim 7, further comprising one or more components selected from the group consisting of a nucleic acid sequence which encodes a second luciferase enzyme and a nucleic acid sequence which encodes an L-cysteine racemase enzyme.

10. The composition of claim 7, wherein the composition comprises of a single vector.

11. The composition of claim 7, wherein the composition comprises more than one vector which may be used in co-transformation of a cell.

12. A composition comprising a cell transformed with the composition of claim 7.

13. The cell according to claim 12, wherein the cell mammalian or plant cell.

14. The method of claim 1 wherein the luciferin recycling protein has an amino acid sequence selected from the group consisting of SEQ ID NO 1, SEQ ID NO 62 or SEQ ID NO 63 or a fragment, or variant thereof.

15. The method of claim 1 wherein the luciferin recycling protein has an amino acid sequence selected from the group consisting of SEQ ID NO 3, SEQ ID NO 39, SEQ ID NO 59, or SEQ ID NO 61, or a fragment or variant thereof.

16. The method of claim 15, wherein the luciferin recycling protein is comprises amino acid SEQ ID NO 59, or a fragment or variant thereof.

17. A composition comprising a luciferin recycling protein wherein the luciferin recycling protein is obtainable from a glow-worm species, or a luciferin recycling fragment thereof, or a protein having at least 60% homology thereto.

18. The composition of claim 17 wherein the glow-worm species is Lampyris noctiluca.

19. The composition of of claim 17, wherein the luciferin recycling protein comprises an amino acid sequence selected from the group consisting of SEQ ID NO 3, SEQ ID NO 39, or SEQ ID NO 59 or a fragment, or variant thereof.

20. The composition of claim 17, wherein the luciferin recycling protein comprises an amino acid sequence selected from the group consisting of SEQ ID NO 3 or SEQ ID NO 39 or a fragment or variant thereof.

21. The composition of claim 17, wherein the luciferin recycling protein comprises amino acid SEQ ID NO 59 or a luciferin recycling fragment, or variant thereof.

22. The composition of claim 21 wherein the luciferin recycling protein comprises amino acid SEQ ID NO 59.

23. A composition comprising a chimeric luciferase recycling protein comprising fragments encoded by at least one exon found in luciferase recycling protein genes of different species.

24. The composition of claim 23, wherein the fragments encoded by at least one exon are from a gene of a glow-worm species.

25. The composition of claim 23, wherein the fragments encoded by at least one exon are from a gene of a firefly species.

26. The composition claim 23, wherein the fragments encoded by at least one exon comprise a fragment encoded by exon 1 of Photinus pyralis luciferase recycling gene, linked to a fragment encoded by exons 2, 3, 4 and 5 of a Lampyris noctiluca luciferase recycling gene.

27. A composition comprising a fusion protein, wherein the fusion protein comprises luciferase enzyme and a luciferin recycling protein.

28. The composition of claim 27, wherein the luciferin recycling protein comprises a luciferin recycling protein obtainable from a glow-worm species, or a luciferin recycling fragment thereof, or a protein having at least 60% homology thereto.

29. A composition comprising a nucleic acid, wherein the nucleic acid encodes luciferin recycling protein of claim 17.

30. The composition of claim 29, wherein the nucleic acid comprises SEQ ID NO 4, SEQ ID NO 31, SEQ ID NO 32, SEQ ID NO 33, SEQ ID NO 34, SEQ ID NO 35, SEQ ID NO 36, or SEQ ID NO 37.

31. The composition of claim 29, wherein the nucleic acid comprises SEQ ID NO 40 or a variant thereof.

32. The composition of claim 29, wherein the nucleic acid comprises SEQ ID NO 58 or a variant thereof.

33. The composition of claim 29, wherein the nucleic acid is a cDNA sequence.

34. A method for producing an optically active enzyme substrate, which comprises contacting an oxidised form of said substrate, with a recycling protein according to claim 17 and an amino acid.

35. The method of claim 1, wherein the method is used to monitor luciferin recycling gene expression.

36. The composition of claim 27, wherein the luciferin recycling protein comprises a chimeric luciferase recycling protein comprising fragments encoded by at least one exon found in luciferase recycling protein genes of different species.

37. A composition comprising a nucleic acid which encodes the luciferin recycling protein of claim 23.

38. A composition comprising a nucleic acid which encodes the fusion protein of claim 27.

39. A method for producing an optically active enzyme substrate, which comprises contacting an oxidised form of said substrate, with a recycling protein according to claim 23 and an amino acid.
